(19) United States
(12) Patent Application Publication
(10) Pub. No.: US 2024/0059728 A1
(43) Pub. Date: Feb. 22, 2024

(54) NOVEL GALACTOSIDE INHIBITOR OF GALECTINS

(71) Applicant: GALECTO BIOTECH AB, Copenhagen N (DK)

(72) Inventors: Kristoffer PETERSON, Copenhagen N (DK); Fredrik ZETTERBERG, Copenhagen N (US); Ulf NILSSON, Copenhagen N (DK)

(73) Assignee: GALECTO BIOTECH AB, Copenhagen N (DK)

(21) Appl. No.: 18/264,488

(22) PCT Filed: Feb. 7, 2022
(86) PCT No.: PCT/EP2022/052856

§ 371 Date: Aug. 7, 2023

(30) Foreign Application Priority Data
Feb. 10, 2021 (DK) PA202100146
Feb. 11, 2021 (EP) 21156635.1
Apr. 15, 2021 (EP) 21168528.4

(51) Int. Cl. C07H 19/056; C07H 19/052

(52) U.S. Cl. CPC C07H 19/056; C07H 19/052

(57) ABSTRACT

A D-galactopyranose compound of formula (1)

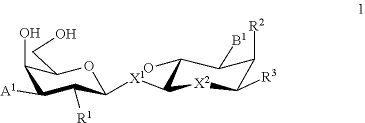

wherein the pyranose ring is beta-D-galactopyranose, and these compounds are high affinity galectin-1 and/or 3 inhibitors for use in treatment of inflammation; fibrosis; scarring; keloid formation; aberrant scar formation; surgical adhesions; scleroderma; systemic sclerosis; septic shock; cancer; metastasising cancers; autoimmune diseases; metabolic disorders; heart disease; heart failure; aortic stenosis; atherosclerosis; pathological angiogenesis; eye diseases; metabolic diseases; insulin resistance; obesity; Diastolic HF; asthma; otosclerosis; mesothelioma; liver disorders Liver cancer; cholangiocarcinoma; biliary tract cancer; and neurodegenerative disorders.

### TECHNICAL FIELD

The present invention relates to novel compounds, the use of said compounds as medicament and for the manufacture of a medicament for the treatment of diseases or disorders such as but not limited to cancers; fibrosis; scarring; keloid formation; aberrant scar formation; surgical adhesions; pathological angiogenesis; eye diseases; HIV-1 diseases; inflammation or transplant rejection in mammals. The invention also relates to pharmaceutical compositions comprising said novel compounds.

### BACKGROUND ART

Galectins are proteins with a characteristic carbohydrate recognition domain (CRD). This is a tightly folded β-sandwich of about 130 amino acids (about 15 kDa) with the two defining features 1) a β-galactose binding site and 2) sufficient similarity in a sequence motif of about seven amino acids, most of which (about six residues) make up the β-galactose binding site. Galectins are synthesized as cytosolic proteins from where they can be targeted to the nucleus, specific cytososlic sites, or secreted to engage in mechanisms effecting physiological functions such as inflammation, immune responses, cell-migration and autophagy. (Johannes et. al 2018) There are now over 9319 publications on galectins in PubMed, with most, as mentioned above, about galectins-1 (>1989) and -3 (>4791). Evidence from literature suggests roles for galectins in e.g. fibrosis, inflammation and cancer (Dings et. al., Dub6-Delarosbil et. al 2017).

Galectin-1 is widely expressed in many cell types and tissues (www.proteinatlas.org) being involved in mechanisms such as apoptosis, adhesion and migration, cell transformation, invasion and metastasis immune escape and angiogenesis. Upregulation of galectin 1 has also been associated with cancer (Dings et. al. 2018), inflammation (Sundblad et. al., 2017) fibrotic disease (Kathiriya et. al 2017, Wu et. al. 2019 and Bennet et. al 2019) and diabetes (Drake et. al. 2022). Example of small molecule ligands including β-D-galactopyranoside were recently reviewed and examplified in Blanchard et. al 2016 and Sethi et. al 2021).

Galectin-3 is widely expressed in many cell types and tissues (www.proteinatlas.org) being involved in mechanisms such as apoptosis, adhesion and migration, cell transformation, invasion and metastasis immune escape and angiogenesis. Upregulation of galectin 3 has also been associated with cancer, inflammation, neurodegenerative disease, fibrotic disease and diabetes (Dings et. al. 2018, Slack et. al. 2020, Li et. al. 2016) Example of small molecule ligands including β-D-galactopyranoside were recently reviewed and examplified in Blanchard et. al 2014 and Sethi et. al 2021.

### SUMMARY OF THE INVENTION

The compounds of the present invention are novel β-D-galactopyranose compounds that unexpectedly have shown high affinity for galectin-1 and/or -3 and are considered novel potent drug candidates.

In broad first aspect the present invention concerns a β-D-galactopyranose compound of formula (1)

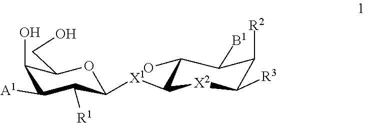

wherein
the pyranose ring whereto A¹ and R¹ is attached is β-D-galactopyranose,
A¹ is (R⁴)ₙ—Z¹,

### Wherein

Z¹ is a five membered heterocycle having at least one heteroatom selected from O, S, and N and is attached to the C3 position of the β-D-galactopyranose;
n is 1 or 2,
each R⁴ is independently selected from
a) an aryl, such as phenyl or naphthyl, optionally substituted with a group selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl;
b) a heterocycle, such as heteroaryl or heterocycloalkyl, optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl;
c) a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen;
d) a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen;
e) Y—W wherein
Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and
W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen;
B¹ is (R^{4a})ₘ—Z²,

Wherein
Z² is a five membered heterocycle having at least one heteroatom selected from O, S, and N and is attached to the D-galactopyranose or the cyclohexyl ring;
m is 1 or 2,
each R^{4a} is independently selected from
a) an aryl, such as phenyl or naphthyl, optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl;
b) a heterocycle, such as heteroaryl or heterocycloalkyl, optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{12a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl;
c) a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen;
d) a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen;
e) Y^{a}—W^{a} wherein
Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen;
X¹ is selected from the group consisting of S, Se, SO, SO₂, O, C═O, and CR¹⁷R¹⁸ wherein R¹⁷ and R¹⁸ are independently selected from hydrogen, OH, or halogen, or X¹ is X^{1a}—X^{1b} wherein X^{1a} and X^{1b} are independently selected from the group consisting of S, Se, SO, SO₂, O, C═O, and CR¹⁹R²⁰ wherein R¹⁹ and R²⁰ are independently selected from hydrogen, OH, or halogen;
X² is O or CH₂;
R¹ is selected from the group consisting of a) H, b) OH, c) OC₁₋₆ alkyl optionally substituted with one or more halogen, phenyl, phenyl substituted with one or more groups selected form OH and halogen, CN, OR²¹, NR²²R²³, and CONH₂, wherein R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, and R²³ is selected from the group consisting of H, CN, a halogen, methyl optionally substituted with a F, OCH₃ optionally substituted with a F, OCH₂CH₃ optionally substituted with a F, OH, and R²⁶—CONH— wherein R²⁶ is selected from C₁₋₃ alkyl and cyclopropyl, d) branched OC₃₋₆ alkyl optionally substituted with one or more halogen, CN, OR²⁷, NR²⁸R²⁹, and CONH₂, wherein R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R³⁰ is selected from C₁₋₃ alkyl and cyclopropyl, R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, and R²⁹ is selected from the group consisting of H, CN, a halogen, methyl optionally substituted with a F, OCH₃ optionally substituted with a F, OCH₂CH₃ optionally substituted with a F, OH, and R³²—CONH— wherein R³² is selected from C₁₋₃ alkyl and cyclopropyl, e) cyclic OC₃₋₆ alkyl optionally substituted with one or more halogen, CN, OR³³, NR³⁴R³⁵, and CONH₂, wherein R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and R³⁵ is selected from the group consisting of H, CN, a halogen, methyl optionally substituted with a F, OCH₃ optionally substituted with a F, OCH₂CH₃ optionally substituted with a F, OH, and R³⁸—CONH— wherein R³⁸ is selected from C₁₋₃ alkyl and cyclopropyl, and
f) a fluorine (F);
R² is selected from H or OH
R³ is selected from H or CH₂OH; or
a pharmaceutically acceptable salt or solvate thereof.

In an embodiment A¹ is

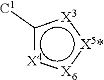

wherein the asterix * indicates the X⁵ atom of the five membered heterocycle that is covalently attached to the galactopyranose,
X³, X⁴, and X⁶ are independently selected from CH, N, O and S, provided that the five membered heterocycle is aromatic,
X⁵ is carbon or nitrogen,
C¹ is a) a phenyl, optionally substituted with a group selected from a halogen; CN; —COOH; —CONR³⁹R⁴⁰, wherein R³⁹ and R⁴⁰ are independently selected from H, C₁₋₃ alkyl and cyclopropyl, or R³⁹ and R⁴⁰ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴¹R⁴², wherein R⁴¹ and R⁴² are independently selected from H and C₁₋₃ alkyl; OH; and R⁴³—CONH— wherein R⁴³ is selected from C₁₋₃ alkyl and cyclopropyl; or b) a heterocycle, such as heteroaryl or heterocycloalkyl, optionally substituted with a group selected from a halogen; a spiro heterocycle; CN; —COOH; —CONR⁴⁴R⁴⁵, wherein R⁴⁴ and R⁴⁵ are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴⁶R⁴⁷, wherein R⁴⁶ and R⁴⁷ are independently selected from H, C₁₋₃ alkyl and cyclopropyl, C(═O)—R⁴⁸, wherein R⁴⁸ is selected from H and C₁₋₃ alkyl; OH; and R⁴⁹—CONH— wherein R⁴⁹ is selected from C₁₋₃ alkyl and cyclopropyl. Typically, A¹ is

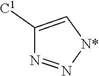

wherein the asterix * indicates the nitrogen atom of the triazole ring that is covalently attached to the galactopyranose; and
C¹ is as defined in the above aspects and embodiments.

In a further embodiment A₁ is

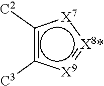

wherein the asterix * indicates the X⁸ atom of the five membered heterocycle that is covalently attached to the galactopyranose,
X⁷ and X⁹ are independently selected from CH, N, O and S, provided that the five membered heterocycle is aromatic,
X⁸ is CH or N,
C² is a) a phenyl, optionally substituted with a group selected from a halogen; CN; —COOH; —CONR^{39a}R^{40a}, wherein R^{39a} and R^{40a} are independently selected from H, C₁₋₃ alkyl and cyclopropyl, or R^{39a} and R^{40a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41a}R^{42a}, wherein R^{41a} and R^{42a} are independently selected from H and C₁₋₃ alkyl; OH; and R^{43a}—CONH— wherein R^{43a} is selected from C₁₋₃ alkyl and cyclopropyl; or b) a heterocycle, such as heteroaryl or heterocycloalkyl, optionally substituted with a group selected from a halogen; a spiro heterocycle; CN; —COOH; —CONR^{44a}R^{45a}, wherein R^{44a} and R^{45a} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46a}R^{47a}, wherein R^{46a} and R^{47a} are independently selected from H, C₁₋₃ alkyl and cyclopropyl, C(═O)—R^{48a}, wherein R^{48a} is selected from H and C₁₋₃ alkyl; OH; and R^{49a}—CONH— wherein R^{49a} is selected from C₁₋₃ alkyl and cyclopropyl; and
C³ is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen;
c) Y—W wherein
Y is linked to the 5 membered heterocycle of A¹ and is selected from the group consisting of S, Se, SO, SO₂, O, C═O, and CR⁶²R⁶³ wherein R⁶² and R⁶³ are independently selected from hydrogen, OH, or halogen; and
W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen. Typically, A¹ is

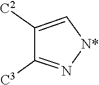

wherein the asterix * indicates the nitrogen atom of the pyrazole ring that is covalently attached to the galactopyranose; and C² and C³ are as defined in the above aspects and embodiments.

In a still further embodiment B¹ is

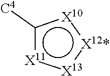

wherein the asterix * indicates the X¹² atom of the five membered heterocycle that is covalently attached to the galactopyranose or cyclohexyl ring;
X¹⁰, X¹¹ and X¹³ are independently selected from CH, N, O and S, provided that the five membered heterocycle is aromatic,
X¹² is CH or N,
C⁴ is a) a phenyl, optionally substituted with a group selected from a halogen; CN; —COOH; —CONR^{39b}R^{40b}, wherein R^{39b} and R^{40b} are independently selected from H, C₁₋₃ alkyl and cyclopropyl, or R^{39b} and R^{40b} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41b}R^{42b}, wherein R^{41b} and R^{42b} are independently selected from H and C₁₋₃ alkyl; OH; and R^{43b}—CONH— wherein R^{43b} is selected from C₁₋₃ alkyl and cyclopropyl; or b) a heterocycle, such as heteroaryl or heterocycloalkyl, optionally substituted with a group selected from a halogen; a spiro heterocycle; CN; —COOH; —CONR^{44b}R^{45b}, wherein R^{44b} and R^{45b} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46b}R^{47b}, wherein R^{46b} and R^{47b} are independently selected from H, C₁₋₃ alkyl and cyclopropyl, C(═O)—R^{48b}, wherein R^{48b} is selected from H and C₁₋₃ alkyl; OH; and R^{49b}—CONH— wherein R^{49b} is selected from C₁₋₃ alkyl and cyclopropyl.

In a further embodiment B¹ is

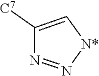

wherein the asterix * indicates the nitrogen atom of the triazole ring that is covalently attached to the galactopyranose or cyclohexyl ring; and
C⁷ is as defined for R^{4a} in the above aspects and embodiments.

In a further embodiment B¹ is

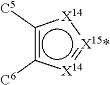

wherein the asterix * indicates the X¹⁵ atom of the five membered heterocycle that is covalently attached to the galactopyranose or cyclohexyl ring;
X¹⁴ and X¹⁶ are independently selected from CH, N, O and S, provided that the five membered heterocycle is aromatic,
X¹⁵ is CH or N,
C⁵ is selected from
a) a phenyl, optionally substituted with a group selected from a halogen; CN; —COOH; —CONR^{39c}R^{40c}, wherein R^{39c} and R^{40c} are independently selected from H, C₁₋₃ alkyl and cyclopropyl, or R^{39c} and R^{40c} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41c}R^{42c}, wherein R^{41c} and R^{42c} are independently selected from H and C₁₋₃ alkyl; OH; and R^{43c}—CONH— wherein R^{43c} is selected from C₁₋₃ alkyl and cyclopropyl; or
b) a heterocycle, such as heteroaryl or heterocycloalkyl, optionally substituted with a group selected from a halogen; a spiro heterocycle; CN; —COOH; —CONR^{44c}R^{45c}, wherein R^{44c} and R^{45c} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46c}R^{47c}, wherein R^{46c} and R^{47c} are independently selected from H, C₁₋₃ alkyl and cyclopropyl, C(═O)—R^{48c}, wherein R^{48c} is selected from H and C₁₋₃ alkyl; OH; and R^{49c}—CONH— wherein R^{49c} is selected from C₁₋₃ alkyl and cyclopropyl; and
C⁶ is selected from
a) a H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen;
b) a phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen;
c) Y^{a}—W^{a} wherein
Y^{a} is linked to the 5 membered heterocycle of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a still further embodiment B¹ is

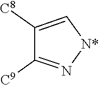

wherein the asterix * indicates the nitrogen atom of the pyrazole ring that is covalently attached to the galactopyranose or cyclohexyl ring; and
C⁸ is as defined is for R^{4a} in the above aspects and embodiments, and
C⁹ is as defined for R^{4a} in the above aspects and embodiments.

In a still further embodiment X¹ is selected from the group consisting of S, Se, SO, SO₂, O, C═O, or X¹ is X^{1a}—X^{1b} wherein X^{1a} and X^{1b} are independently selected from the group consisting of S, Se, SO, SO₂, O, C═O.

In a further embodiment X² is O. Alternatively, X² is CH₂.

In a still further embodiment R¹ is selected from the group consisting of a) H, b) OH, c) OC₁₋₄ alkyl, d) branched OC₃₋₄ alkyl, and e) cyclic OC₃₋₄ alkyl.

In a further embodiment R² is H. Alternatively, R² is OH.

In a still further embodiment R³ is H. Alternatively, R³ is CH₂OH.

In a further embodiment the compound of formula (1) is selected from any one of the group consisting of:
3,3′-Dideoxy-3-[4-(3,4,5-trifluorophenyl)-1H-1,2,3-triazol-1-yl]-3′-[4-(2-hydroxythiazol-4-yl)-1H-1,2,3-triazol-1-yl]-1,1′-sulfanediyl-di-β-D-galactopyranoside,
3-[4-(4-Chloro-3,5-difluorophenyl)-1H-1,2,3-triazol-1-yl]-3,3′-dideoxy-3′-[4-(2-hydroxythiazol-4-yl)-1H-1,2,3-triazol-1-yl]-1,1′-sulfanediyl-di-β-D-galactopyranoside,
3′-[4-(2-Aminothiazol-4-yl)-1H-1,2,3-triazol-1-yl]-3-[4-(4-chloro-3,5-difluorophenyl)-1H-1,2,3-triazol-1-yl]-3,3′-dideoxy-1,1′-sulfanediyl-di-β-D-galactopyranoside,
   1,1′-Sulfanediyl-bis-{3-deoxy-3-[4-(3-fluorophenyl)-1H-1,2-pyrazol-1-yl]-β-D-galactopyranoside},
   1,1′-Sulfanediyl-bis-{3-deoxy-3-[4-(3,4,5-trifluorophenyl)-1H-1,2-pyrazol-1-yl]-β-D-galactopyranoside},
   3,3′-Dideoxy-3-[4-(3,4,5-trifluorophenyl)-1H-1,2-pyrazol-1-yl]-3′-[4-(2-hydroxythiazol-4-yl)-1H-1,2,3-triazol-1-yl]-1,1′-sulfanediyl-di-β-D-galactopyranoside,
   3,3′-Dideoxy-3-[4-(3,4,5-trifluorophenyl)-1H-1,2-pyrazol-1-yl]-3′-[4-(morpholin-4-yl)-1H-1,2-pyrazol-1-yl]-1,1′-sulfanediyl-di-β-D-galactopyranoside,
   3,3′-Dideoxy-3-[4-(3,4,5-trifluorophenyl)-1H-1,2-pyrazol-1-yl]-3′-(4-isopropyl-1H-1,2-pyrazol-1-yl)-1,1′-sulfanediyl-di-β-D-galactopyranoside,
   (2S,3R,4S)-3-Hydroxy-4-[4-(3,4,5-trifluorophenyl)-1H-1,2-pyrazol-1-yl]tetrahydropyran-2-yl 3-deoxy-3-[4-(3,4,5-trifluorophenyl)-1H-1,2-pyrazol-1-yl]-2-O-methyl-1-thio-β-D-galactopyranoside,
(1R,2R,6S)-1-Hydroxy-6-[4-(3,4,5-trifluorophenyl)-1H-1,2-pyrazol-1-yl]cyclohexan-2-yl 3-deoxy-3-[4-(3,4,5-trifluorophenyl)-1H-1,2-pyrazol-1-yl]-2-O-methyl-1-thio-β-D-galactopyranoside,
3-Deoxy-3-[4-(3-fluorophenyl)-1H-1,2-pyrazol-1-yl]-β-D-galactopyranosyl 3-deoxy-3-[4-(3-fluorophenyl)-1H-1,2-pyrazol-1-yl]-β-D-galactopyranoside,
1,1′-Selenediyl-bis-{3-deoxy-3-[4-(3-fluorophenyl)-1H-1,2-pyrazol-1-yl]-β-D-galactopyranoside}, and
Bis-{3-deoxy-3-[4-(3-fluorophenyl)-1H-1,2-pyrazol-1-yl]-β-D-galactopyranos-1-yl} disulfide; or a pharmaceutically acceptable salt or solvate thereof.

In a further aspect the present invention relates to a compound of formula (1) for use as a medicine.

In a still further aspect, the present invention relates to a pharmaceutical composition comprising the compound of any one of the previous claims and optionally a pharmaceutically acceptable additive, such as a carrier and/or excipient.

In a further aspect the present invention relates to a compound of formula (1) of the present invention for use in a method for treating a disorder relating to the binding of a galectin-1 and/or -3 to a ligand in a mammal, such as a human. In a further embodiment the disease or disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; scleroderma; systemic sclerosis; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistance; obesity; Diastolic HF; asthma and other interstitial lung diseases, including Hermansky-Pudlak syndrome, pulmonary arterial hypertension, RA-ILD, SSc-ILD, Lung disease with fibrosis such as COPD and asthma. Otosclerosis, mesothelioma; liver disorders, such as non-alcoholic steatohepatitis or non-alcoholic fatty liver disease, Liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease. Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis, multiple sclerosis, peripheral nephropathy.

In a still further aspect the present invention relates to a method for treatment of a disease or disorder relating to the binding of a galectin-1 and/or -3 to a ligand in a mammal, such as a human, wherein a therapeutically effective amount of at least one compound of formula (1) of the present invention is administered to a mammal in need of said treatment.

In a further embodiment the disease or disorder is selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; scleroderma; systemic sclerosis; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistance; obesity; Diastolic HF; asthma and other interstitial lung diseases, including Hermansky-Pudlak syndrome, pulmonary arterial hypertension, RA-ILD, SSc-ILD, Lung disease with fibrosis such as COPD and asthma. Otosclerosis, mesothelioma; liver disorders, such as non-alcoholic steatohepatitis or non-alcoholic fatty liver disease, Liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease. Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis, multiple sclerosis, peripheral nephropathy.

Another aspect of the present invention concerns combination therapy involving administering a compound of formula (I) of the present invention together with a therapeutically active compound different from the compound of formula (I) (interchangeable with “a different therapeutically active compound”). In one embodiment the present invention relates to a combination of a compound of formula (I) and a different therapeutically active compound for use in treatment of a disorder relating to the binding of a galectin-1 and/or -3 to a ligand in a mammal. Such disorders are disclosed below.

In an embodiment of the present invention, a therapeutically effective amount of at least one compound of formula (I) of the present invention is administered to a mammal in need thereof in combination with a different therapeutically active compound. In a further embodiment, said combination of a compound of formula (I) together with a different therapeutically active compound is administered to a mammal suffering from a disorder selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis or non-alcoholic fatty liver disease.

A non-limiting group of cancers given as examples of cancers that may be treated, managed and/or prevented by administration of a compound of formula (I) in combination with a different therapeutically active compound is selected from: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In some aspects of the present invention, the administration of at least one compound of formula (I) of the present invention and at least one additional therapeutic agent demonstrates therapeutic synergy. In some aspects of the methods of the present invention, a measurement of response to treatment observed after administering both at least one compound of formula (I) of the present invention and the additional therapeutic agent is improved over the same measurement of response to treatment observed after administering either the at least one compound of formula (I) of the present invention or the additional therapeutic agent alone.

A further aspect of the present invention concerns combination therapy involving administering a compound of formula (I) of the present invention together with an anti-fibrotic compound different form the compound of formula (I) to a mammal in need thereof. In a further embodiment, such anti-fibrotic compound may be selected from the following non-limiting group of anti-fibrotic compounds: pirfenidone, nintedanib, simtuzumab (GS-6624, AB0024), BG00011 (STX100), PRM-151, PRM-167, PEG-FGF21, BMS-986020, FG-3019, MN-001, IW001, SAR156597, GSK2126458, PAT-1251 and PBI-4050.

A still further aspect of the present invention concerns combination therapy involving administering a compound of formula (I) in combination with a further conventional cancer treatment such as chemotherapy or radiotherapy, or treatment with immunostimulating substances, gene therapy, treatment with antibodies and treatment using dendritic cells, to a mammal in need thereof.

In an embodiment the compound of formula (I) is administered together with at least one additional therapeutic agent selected from an antineoplastic chemotherapy agent. In a further embodiment, the antineoplastic chemotherapeutic agent is selected from: all-trans retinoic acid, Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

In a further embodiment of the present invention, the further conventional cancer treatment includes radiation therapy. In some embodiments, radiation therapy includes localized radiation therapy delivered to the tumor. In some embodiments, radiation therapy includes total body irradiation.

In other embodiments of the present invention the further cancer treatment is selected from the group of immunostimulating substances e.g. cytokines and antibodies. Such cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40 or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In some embodiments of the present invention the compound of formula (I) is administered together with at least one additional therapeutic agent selected from a checkpoint inhibitor. In some embodiments of the invention, the checkpoint inhibitor is acting on one or more of the following, non-limiting group of targets: CEACAM1, galectin-9, TIM3, CD80, CTLA4, PD-1, PD-L1, HVEM, BTLA, CD160, VISTA, B7-H4, B7-2, CD155, CD226, TIGIT, CD96, LAG3, GITF, OX40, CD137, CD40, IDO, and TDO. These are known targets and some of these targets are described in Melero et al., Nature Reviews Cancer (2015). Examples of check point inhibitors administered together with the compound of formula (1) are Anti-PD-1: Nivolumab, Pembrolizumab, Cemiplimab. Anti-PD-L1: Atezolizumab, Avelumab, Durvalumab and one Anti-CTLA-4: Ipilimumab. Each one of these check point inhibitors can be made the subject of an embodiment in combination with any one of the compounds of formula (1).

In some embodiments of the present invention the compound of formula (I) is administered together with at least one additional therapeutic agent selected from an inhibitor of indoleamine-2,3-dioxygenase (IDO).

In some embodiments of the present invention the compound of formula (I) is administered together with at least one additional therapeutic agent selected from one or more inhibitors of the CTLA4 pathway. In some embodiments, the inhibitor of the CTLA4 pathway is selected from one or more antibodies against CTLA4.

In some embodiments of the present invention the compound of formula (I) is administered together with at least one additional therapeutic agent selected from one or more inhibitors of the PD-1/PD-L pathway.

In some embodiments, the one or more inhibitors of the PD-1/PD-L pathway are selected from one or more antibodies or antibody fragments against PD-1, PD-L1, and/or PD-L2, or other ways by which an anti-PD1 antibodies can be induced such as mRNA based introduction of genetic material which sets forth in-body production of anti-PD1 or anti-PDL1 antibodies or fragments of such antibodies.

In a still further aspect the present invention relates to a process of preparing a compound of formula II or a pharmaceutically acceptable salt or solvate thereof comprising the step a1 where C¹, C⁴ and X¹ are defined as above under formula 1;

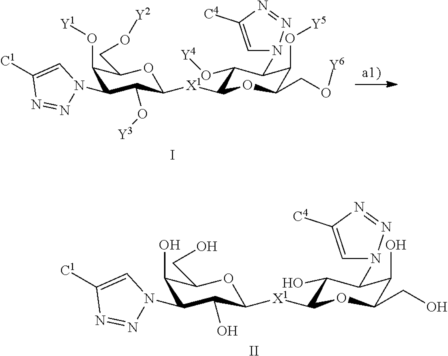

a1) Reacting a compound of the formula I wherein Y¹-Y⁶ are protective groups such as acetates with a base, such as triethylamine, LiOH or sodium methoxide in a suitable solvent, such as methanol and water to provide a compound of the formula II.

In a still further aspect the present invention relates to a process of preparing a compound of formula IV or a pharmaceutically acceptable salt or solvate thereof comprising the step a2 where C¹, C⁴, X¹, X², R¹, R² and R³ are defined as above under formula 1;

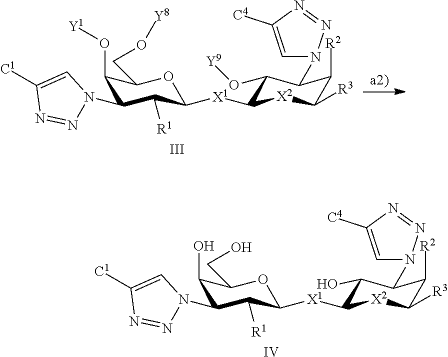

a2) Reacting a compound of the formula III wherein Y⁷, Y⁸ and Y⁹ are protective groups such as acetates with a base, such as triethylamine, LiOH or sodium methoxide in a suitable solvent, such as methanol and water to provide a compound of the formula IV.

In a still further aspect the present invention relates to a process of preparing a compound of formula VI or a pharmaceutically acceptable salt or solvate thereof comprising the step a3 where C², C³, C⁵, C⁶ and X¹ are defined as above under formula 1;

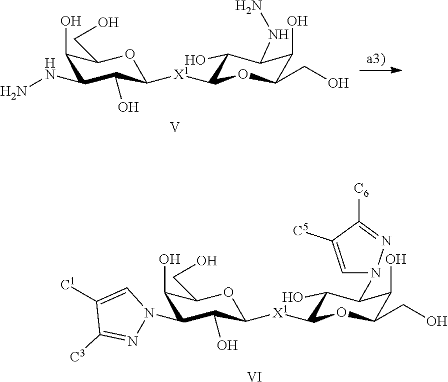

a3) Reacting a compound of the formula V with a compound of formula C³—CO—CC²—CN(Me)₂ in the presence of an acid such as HCl in a solvent such as ethanol optionally at elevated temperature (above room temperature) yielding an intermediate that is further reacted with C⁶—CO—CC⁵—CN(Me)₂ in the presence of an acid such as HCl in a solvent such as ethanol optionally at elevated temperature to give a compound of the formula VI.

In a still further aspect the present invention relates to a process of preparing a compound of formula VIII or a pharmaceutically acceptable salt or solvate thereof comprising the step a4 where C², C³, C⁵, C⁶, X¹, X², R¹, R² and R³ are defined as above under formula 1;

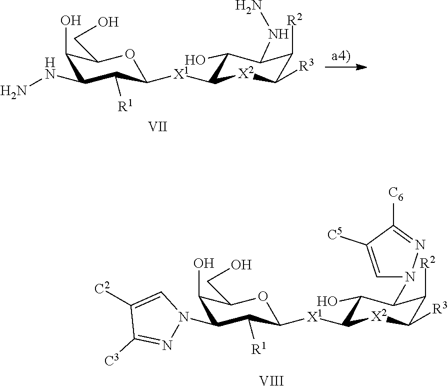

a4) Reacting a compound of the formula VII with a compound of the formula C³—CO—CC²—CN(Me)₂ in the presence of an acid such as HCl in a solvent such as ethanol optionally at elevated temperature (above room temperature) yielding an intermediate that is further reacted with C⁶—CO—CC⁵—CN(Me)₂ in the presence of an acid such as HCl in a solvent such as ethanol optionally at elevated temperature to give a compound of the formula VIII.

In a still further aspect the present invention relates to a process of preparing a compound of formula X or a pharmaceutically acceptable salt or solvate thereof comprising the step a5 where C¹, C⁵, C⁶ and X¹ are defined as above under formula 1;

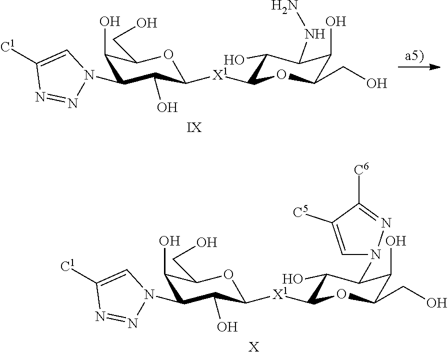

a5) Reacting a compound of the formula IX with a compound of the formula such as C⁶—CO—CC⁵—CN(Me)₂ in the presence of an acid such as HCl in a solvent such as ethanol optionally at elevated temperature to give a compound of the formula X.

In a still further aspect the present invention relates to a process of preparing a compound of formula I or a pharmaceutically acceptable salt or solvate thereof comprising the step a6 where C¹, C⁴ and X¹ are defined as above under formula 1 and Y¹-Y⁶ are protective groups such as acetates;

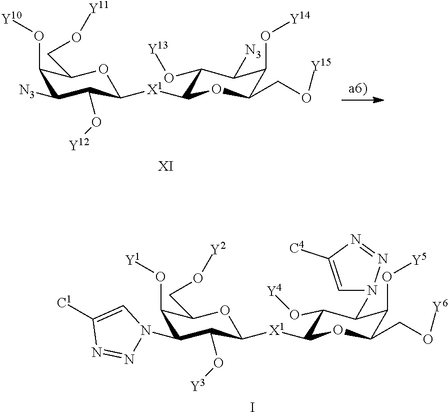

a6) Reacting a compound of the formula XI wherein Y¹⁰-Y¹⁵ are protective groups such as acetates with a compound of the formula C¹—CC—H or C¹—CC-TMS in an inert solvent, such as DMF or acetonitrile, using a base, such as diisopropylethylamine, catalyzed by a cupper salt such as CuI, optionally using a reagent such as CsF, optionally at elevated temperature (above room temperature) yielding an intermediate, which is further reacted with a compound of the formula C⁴—CC—H or C⁴—CC-TMS in an inert solvent, such as DMF or acetonitrile, using a base, such as diisopropylethylamine, catalyzed by a cupper salt such as CuI, optionally using a reagent such as CsF, optionally at elevated temperature to give a compound of the formula I.

In a still further aspect the present invention relates to a process of preparing a compound of formula V or a pharmaceutically acceptable salt or solvate thereof comprising the steps a7-a11 wherein X¹, is defined as above under formula 1;

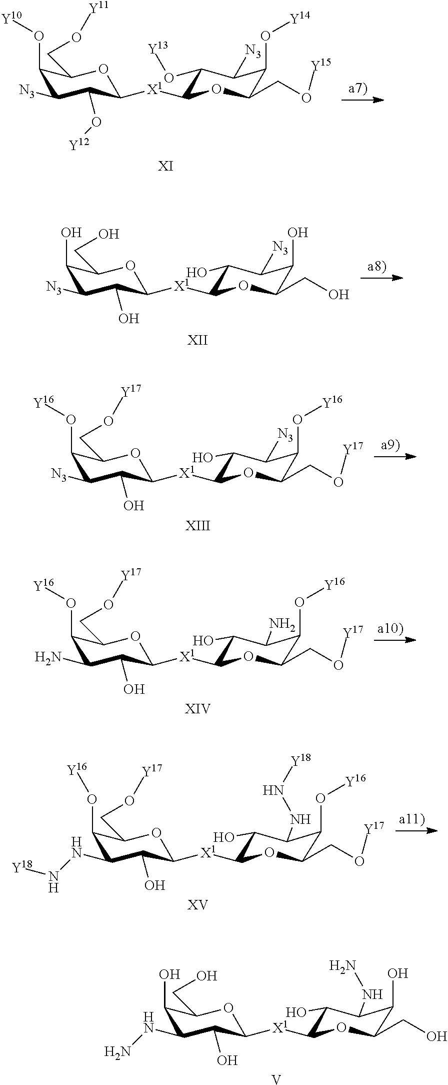

a7) Reacting a compound of the formula XI wherein Y¹⁰-Y¹⁵ are protective groups such as acetates with a base, such as triethylamine, LiOH or sodium methoxide in a suitable solvent, such as methanol and water to give a compound of the formula XII.
a8) Reacting a compound of the formula XII with a reagent such as benzaldehyde dimethyl acetal in the presence of an acid, such as D(+)-10-camphorsulfonic acid, in an inert solvent such as DMF optionally at elevated temperature to give a compound of formula XIII wherein Y¹⁶ and Y¹⁷ together form a protecting group such as benzylidene.
a9) Reacting a compound of the formula XIII with a reducing agent such as triphenylphosphine in an inert solvent such as THF and water optionally at elevated temperature to give a compound of formula XIV.
a10) Reacting a compound of the formula XIV with N-tert-butyloxycarbonyl-3-(4-cyanophenyl)oxaziridine in an inert solvent such as DCM to give a compound of formula XV wherein Y¹⁸ is a protective group such as tert-butyloxycarbonyl.
a11) Reacting a compound of the formula XV with an acid such as TFA in an inert solvent such as DCM to give a compound of formula V.

In a still further aspect, the present invention relates to a process of preparing a compound of formula IX or a pharmaceutically acceptable salt or solvate thereof comprising the steps a12-a17 wherein C¹ and X¹, are defined as above under formula 1;

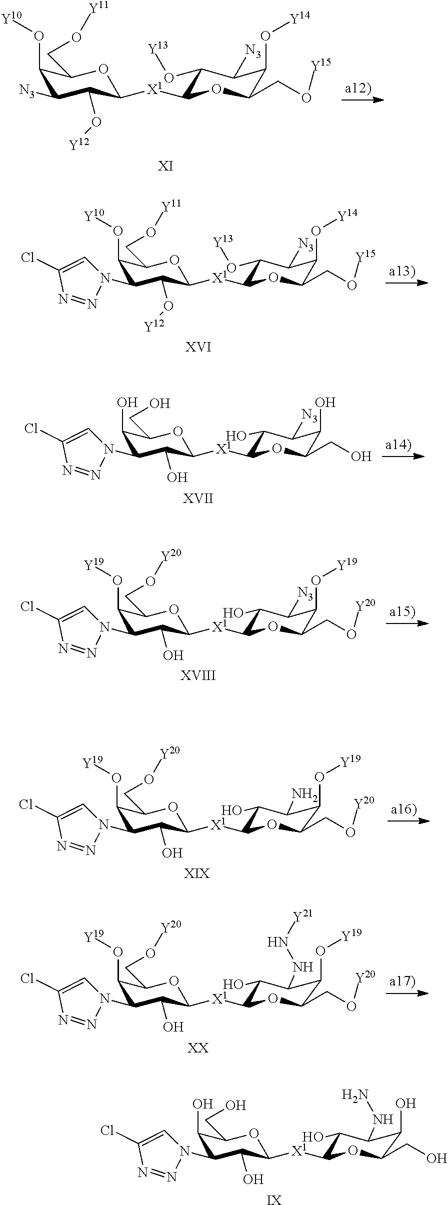

a12) Reacting a compound of the formula XI wherein Y¹⁰-Y¹⁵ are protective groups such as acetates with a compound of formula C¹—CC—H or C¹—CC-TMS in an inert solvent, such as DMF or acetonitrile, using a base, such as diisopropylethylamine, catalyzed by a cupper salt such as CuI, optionally using a reagent such as CsF, optionally at elevated temperature to give a compound of the formula XVI.
a13) Reacting a compound of the formula XVI wherein Y¹⁰-Y¹⁵ are protective groups such as acetates with a base, such as triethylamine, LiOH or sodium methoxide in a suitable solvent, such as methanol and water to give a compound of the formula XVII.
a14) Reacting a compound of the formula XVII with a reagent such as benzaldehyde dimethyl acetal in the presence of an acid such as D(+)-10-camphorsulfonic acid, in an inert solvent such as DMF optionally at elevated temperature to give a compound of formula XVIII wherein Y¹⁹ and Y²⁰ together form a protecting group such as benzylidene.
a15) Reacting a compound of the formula XVIII with a reducing agent such as triphenylphosphine in an inert solvent such as THF and water optionally at elevated temperature to give a compound of formula XIX.
a16) Reacting a compound of the formula XIX with N-tert-butyloxycarbonyl-3-(4-cyanophenyl)oxaziridine in an inert solvent such as DCM to give a compound of formula XX wherein Y²¹ is a protective group such as tert-butyloxycarbonyl.
a17) Reacting a compound of the formula XX with an acid such as TFA in an inert solvent such as DCM to give a compound of formula IX.

In a still further aspect the present invention relates to a process of preparing a compound of formula XXII or a pharmaceutically acceptable salt or solvate thereof comprising the step a18 where C² is defined as above under formula 1;

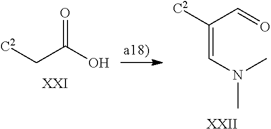

a18) Reacting a compound of the formula XXI with phosphorus oxychloride in a solvent, such as DMF, optionally at elevated temperature, followed by reacting the product with a base, such as sodium carbonate in solvents, such as water and toluene, optionally at elevated temperature to give a compound of the formula XXII.

### DETAILED DESCRIPTION OF THE INVENTION

The present compounds of formula (1) differ from prior art compounds particularly in that the pyranose ring is β-D-galactopyranose. It is important to emphasize that alpha and beta anomers are very different isomers and it is by no means considered to be obvious to the skilled person to expect same or similar activity of both anomers. Consequently, alpha and beta anomers do not in general posses the same activity, and this is common knowledge to the skilled person. The compounds of the present invention are novel β-D-galactopyranose compounds that unexpectedly have shown very high affinity and specificity for galectin-1 and/or -3, and typically have a higher specificity for galectin-3 over galectin-1, and are considered novel potent drug candidates.

In broad aspect the present invention concerns a β-D-galactopyranose compound of formula (1)

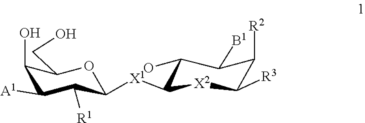

Wherein A¹, R¹, R², R³, X¹, X² and B¹ are as defined in the above aspects and embodiments.

In a first embodiment A¹ is (R⁴)ₙ—Z¹, wherein n is 1.

In a further embodiment, when n is 1, Z¹ is a five membered heterocycle containing 2 or 3 nitrogens, such as a pyrazole or triazole.

In a still further embodiment, when n is 1, R⁴ is a phenyl optionally substituted with a group selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl. Typically, the phenyl is substituted with one, two or three substituents.

In a further embodiment, when n is 1, R⁴ is a heteroaryl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl. Typically, the heteroaryl is substituted with one, two or three substituents.

In a still further embodiment, when n is 1, R⁴ is a heterocycloalkyl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl. Typically, the heterocycloalkyl is substituted with one, two or three substituents.

In a further embodiment, when n is 1, R⁴ is a group selected from Y—W wherein Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

In a further embodiment A¹ is

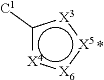

wherein the asterix * indicates the X⁵ atom of the five membered heterocycle that is covalently attached to the galactopyranose, and C¹, X³, X⁴, X⁵ and X⁶ are as defined in the above aspects and embodiments.

In an embodiment X⁵ is selected from CH or N, typically N.

In a further embodiment X³ is selected from CH, N, O and S, typically CH and N.

In a further embodiment X⁴ is selected from CH, N, O and S, typically CH and N.

In a further embodiment X⁶ is selected from CH, N, O and S, typically CH and N.

In a further embodiment C¹ is a phenyl, optionally substituted with a group selected from a halogen; CN; —COOH; —CONR³⁹R⁴⁰, wherein R³⁹ and R⁴⁰ are independently selected from H, C₁₋₃ alkyl and cyclopropyl, or R³⁹ and R⁴⁰ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴¹R⁴², wherein R⁴¹ and R⁴² are independently selected from H and C₁₋₃ alkyl; OH; and R⁴³—CONH— wherein R⁴³ is selected from C₁₋₃ alkyl and cyclopropyl.

In another embodiment C¹ is a heteroaryl optionally substituted with a group selected from a halogen; a spiro heterocycle; CN; —COOH; —CONR⁴¹R⁴⁵, wherein R⁴ and R⁴⁵ are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴⁶R⁴⁷, wherein R⁴⁶ and R⁴⁷ are independently selected from H, C₁₋₃ alkyl and cyclopropyl, C(═O)—R⁴⁸, wherein R⁴⁸ is selected from H and C₁₋₃ alkyl; OH; and R⁴⁹—CONH— wherein R⁴⁹ is selected from C₁₋₃ alkyl and cyclopropyl.

In a further embodiment C¹ is a heterocycloalkyl optionally substituted with a group selected from a halogen; a spiro heterocycle; CN; —COOH; —CONR⁴⁴R⁴⁵, wherein R⁴⁴ and R⁴⁵ are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴⁶R⁴⁷, wherein R⁴⁶ and R⁴⁷ are independently selected from H, C₁₋₃ alkyl and cyclopropyl, C(═O)—R⁴⁸, wherein R⁴⁸ is selected from H and C₁₋₃ alkyl; OH; and R⁴⁹—CONH— wherein R⁴⁹ is selected from C₁₋₃ alkyl and cyclopropyl.

Typically, A¹ is

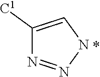

wherein the asterix * indicates the nitrogen atom of the triazole ring that is covalently attached to the galactopyranose; and
C¹ is a phenyl substituted with one, two or three substituents selected from halogen, such as a phenyl substituted with three substituents selected from F and Cl.

In a second embodiment A¹ is (R⁴)ₙ—Z¹, wherein n is 2.

In a further embodiment, when n is 2, Z¹ is a five membered heterocycle containing 2 or 3 nitrogens, such as a pyrazole or triazole.

In a still further embodiment, when n is 2, one R⁴ is a phenyl optionally substituted with a group, such as one, two or three substituents, selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl; and the second R⁴ is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen.

In a further embodiment, when n is 2, one R⁴ is a phenyl optionally substituted with a group, such as one, two or three substituents, selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl; and the second R⁴ is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen.

In a further embodiment, when n is 2, one R⁴ is a phenyl optionally substituted with a group, such as one, two or three substituents, selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl; and the second R⁴ is a group selected from Y—W wherein

Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and

W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

In a further embodiment, when n is 2, one R⁴ is a heteroaryl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen.

In a further embodiment, when n is 2, one R⁴ is a heteroaryl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen.

In a further embodiment, when n is 2, one R⁴ is a heteroaryl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from Y—W wherein

Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and

W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

In a further embodiment, when n is 2, one R⁴ is a heterocycloalkyl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen.

In a further embodiment, when n is 2, one R⁴ is a heterocycloalkyl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen.

In a further embodiment, when n is 2, one R⁴ is a heterocycloalkyl optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl; and the second R⁴ is a group selected from Y—W wherein

Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and

W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

In a further embodiment A¹ is

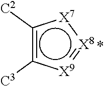

wherein the asterix * indicates the X⁸ atom of the five membered heterocycle that is covalently attached to the galactopyranose, and C², C³, X⁷, X⁸, and X⁹ are as defined in the above aspects and embodiments.

In an embodiment X⁸ is CH or N, typically N.

In a further embodiment X⁷ is selected from CH, N, O and S, typically CH and N.

In a still further embodiment X⁹ is selected from CH, N, O and S, typically CH and N.

In a further embodiment C² is a phenyl, optionally substituted with a group selected from a halogen; CN; —COOH; —CONR^{39a}R^{40a}, wherein R^{39a} and R^{40a} are independently selected from H, C₁₋₃ alkyl and cyclopropyl, or R^{39a} and R^{40a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41a}R^{42a}, wherein R^{41a} and R^{42a} are independently selected from H and C₁₋₃ alkyl; OH; and R^{43a}—CONH— wherein R^{43a} is selected from C₁₋₃ alkyl and cyclopropyl.

In a still further embodiment C² is a heteroaryl optionally substituted with a group selected from a halogen; a spiro heterocycle; CN; —COOH; —CONR^{44a}R^{45a}, wherein R^{44a} and R^{45a} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46a}R^{47a}, wherein R^{46a} and R^{47a} are independently selected from H, C₁₋₃ alkyl and cyclopropyl, C(═O)—R^{48a}, wherein R^{48a} is selected from H and C₁₋₃ alkyl; OH; and R^{49a}—CONH— wherein R^{49a} is selected from C₁₋₃ alkyl and cyclopropyl.

In a further embodiment C² is a heterocycloalkyl optionally substituted with a group selected from a halogen; a spiro heterocycle; CN; —COOH; —CONR^{4a}R^{45a}, wherein R^{44a} and R^{45a} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46a}R^{47a}, wherein R^{46a} and R^{47a} are independently selected from H, C₁₋₃ alkyl and cyclopropyl, C(═O)—R^{48a}, wherein R^{48a} is selected from H and C₁₋₃ alkyl; OH; and R^{49a}—CONH— wherein R^{49a} is selected from C₁₋₃ alkyl and cyclopropyl.

In a still further embodiment C³ is selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen.

In a still further embodiment C³ is selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen.

In a still further embodiment C³ is selected from Y—W wherein Y is linked to the 5 membered heterocycle of A¹ and is selected from the group consisting of S, Se, SO, SO₂, O, C═O, and CR⁶²R⁶³ wherein R⁶² and R⁶³ are independently selected from hydrogen, OH, or halogen; and W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁶⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

Typically, A¹ is

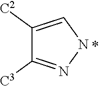

wherein the asterix * indicates the nitrogen atom of the pyrazole ring that is covalently attached to the galactopyranose;
C² is phenyl substituted with one, two or three substitutents selected from halogen; and
C³ is H.

In a first embodiment B¹ is (R^{4a})ₘ—Z², wherein m is 1.

In a further embodiment, when m is 1, Z² is a five membered heterocycle containing 2 or 3 nitrogens, such as a pyrazole or triazole.

In a still further embodiment, when m is 1, R^{4a} is a phenyl optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl. Typically, the phenyl is substituted with one, two or three substituents.

In a still further embodiment, when m is 1, R^{4a} is a heteroaryl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{12a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl. Typically, the heteroaryl is substituted with one, two or three substituents.

In a still further embodiment, when m is 1, R^{4a} is a heterocycloalkyl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl. Typically, the heterocycloalkyl is substituted with one, two or three substituents.

In a further embodiment, when m is 1, R^{4a} is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen.

In a further embodiment, when m is 1, R^{4a} is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen.

In a further embodiment, when m is 1, R^{4a} is a group selected from Y^{a}—W^{a} wherein
Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a still further embodiment B¹ is

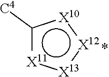

wherein the asterix * indicates the X¹² atom of the five membered heterocycle that is covalently attached to the galactopyranose or cyclohexyl ring.

In a further embodiment X¹⁰ is selected from CH, N, O and S, typically CH and N.

In a still further embodiment X¹¹ is selected from CH, N, O and S, typically CH and N.

In a further embodiment X¹³ is selected from CH, N, O and S, typically CH and N.

In a still further embodiment X¹² is CH or N, preferably N.

In a further embodiment C⁴ is a phenyl, optionally substituted with a group selected from a halogen; CN; —COOH; —CONR^{39b}R^{40b}, wherein R^{39b} and R^{40b} are independently selected from H, C₁₋₃ alkyl and cyclopropyl, or R^{39b} and R^{40b} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41b}R^{42b}, wherein R^{41b} and R^{42b} are independently selected from H and C₁₋₃ alkyl; OH; and R^{43b}—CONH— wherein R^{43b} is selected from C₁₋₃ alkyl and cyclopropyl. Typically, C⁴ is a phenyl substituted with one, two or three groups selected from a halogen.

In a further embodiment C⁴ is a heteroaryl optionally substituted with a group selected from a halogen; a spiro heterocycle; CN; —COOH; —CONR^{44b}R^{45b}, wherein R^{44b} and R^{45b} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46b}R^{47b}, wherein R^{46b} and R^{47b} are independently selected from H, C₁₋₃ alkyl and cyclopropyl, C(═O)—R^{48b}, wherein R^{48b} is selected from H and C₁₋₃ alkyl; OH; and R^{49b}—CONH— wherein R^{49b} is selected from C₁₋₃ alkyl and cyclopropyl. Typically, C⁴ is a heteroaryl, such as a thiazolyl, substituted with one, two or three groups selected from OH and NH₂.

In a further embodiment C⁴ is a heterocycloalkyl optionally substituted with a group selected from a halogen; a spiro heterocycle; CN; —COOH; —CONR^{44b}R^{45b}, wherein R^{44b} and R^{45b} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46b}R^{47b}, wherein R^{46b} and R^{47b} are independently selected from H, C₁₋₃ alkyl and cyclopropyl, C(═O)—R^{48b} wherein R^{48b} is selected from H and C₁₋₃ alkyl; OH; and R^{49b}—CONH— wherein R^{49b} is selected from C₁₋₃ alkyl and cyclopropyl. Typically, C⁴ is a heterocycloalkyl, such as a morpholinyl.

In a further embodiment C⁴ is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen. Typically, C⁴ is a C₁₋₆ alkyl optionally substituted with a halogen, such as a C₁₋₄ alkyl, e.g. isopropyl.

In a second embodiment B¹ is (R^{4a})ₘ—Z², wherein m is 2.

In a further embodiment, when m is 2, Z² is a five membered heterocycle containing 2 or 3 nitrogens, such as a pyrazole or triazole.

In a still further embodiment, when m is 2, one R^{4a} is a phenyl optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl; and the second R^{4a} is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen.

In a still further embodiment, when m is 2, one R^{4a} is a phenyl optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl; and the second R^{4a} is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen.

In a still further embodiment, when m is 2, one R^{4a} is a phenyl optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl; and the second R^{4a} is a group selected from Y^{a}—W^{a} wherein
Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a still further embodiment, when m is 2, one R^{4a} is a heteroaryl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen.

In a still further embodiment, when m is 2, one R^{4a} is a heteroaryl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen.

In a still further embodiment, when m is 2, one R^{4a} is a heteroaryl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from Y^{a}—W^{a} wherein Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a still further embodiment, when m is 2, one R^{4a} is a heterocycloalkyl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen.

In a still further embodiment, when m is 2, one R^{4a} is a heterocycloalkyl optionally substituted with a group selected from a halogen; a spiro heterocycle; CN; —COOH; —CONR^{11a}R^{12a}, wherein R^{11a} and R^{2a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{15a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen.

In a still further embodiment, when m is 2, one R^{4a} is a heterocycloalkyl optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{2a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{5a}, wherein R^{15a} is selected from H and C₁₋₃ alkyl; OH; and R^{16a}—CONH— wherein R^{16a} is selected from C₁₋₃ alkyl and cyclopropyl; and the second R^{4a} is a group selected from Y^{a}—W^{a} wherein
Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a further embodiment B¹ is

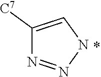

wherein the asterix * indicates the nitrogen atom of the triazole ring that is covalently attached to the galactopyranose or cyclohexyl ring; and
C⁷ is a heteroaryl, such as thiazolyl, optionally substituted with one or two groups selected from OH and NH₂. Typically, C⁷ is a thiazolyl substituted with one group selected from OH and NH₂.

In a further embodiment B¹ is

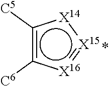

wherein the asterix * indicates the X¹⁵ atom of the five membered heterocycle that is covalently attached to the galactopyranose or cyclohexyl ring.

In an embodiment X¹⁴ is selected from CH, N, O and S, typically CH and N.

In a further embodiment X¹⁶ is selected from CH, N, O and S, typically CH and N.

In a still further embodiment X¹⁵ is CH or N, typically N.

In a further embodiment C⁵ is selected from a phenyl, optionally substituted with a group selected from a halogen; CN; —COOH; —CONR^{39c}R^{40c}, wherein R^{39c} and R^{40c} are independently selected from H, C₁₋₃ alkyl and cyclopropyl, or R^{39c} and R^{40c} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41c}R^{42c}, wherein R^{41c} and R^{42c} are independently selected from H and C₁₋₃ alkyl; OH; and R^{43c}—CONH— wherein R^{43c} is selected from C₁₋₃ alkyl and cyclopropyl.

Typically, C⁵ is selected from a phenyl substituted with one, two or three groups selected from a halogen.

In a still further embodiment C⁵ is selected from a heteroaryl optionally substituted with a group selected from a halogen; a spiro heterocycle; CN; —COOH; —CONR^{44c}R^{45c}, wherein R^{44c} and R^{45c} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46c}R^{47c}, wherein R^{46c} and R^{47c} are independently selected from H, C₁₋₃ alkyl and cyclopropyl, C(═O)—R^{48c}, wherein R^{48c} is selected from H and C₁₋₃ alkyl; OH; and R^{49c}—CONH— wherein R^{49c} is selected from C₁₋₃ alkyl and cyclopropyl.

In a further embodiment C⁵ is selected from a heterocycloalkyl optionally substituted with a group selected from a halogen; a spiro heterocycle; CN; —COOH; —CONR^{44c}R^{45c}, wherein R^{44c} and R^{45c} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46c}R^{47c}, wherein R^{46c} and R^{47c} are independently selected from H, C₁₋₃ alkyl and cyclopropyl, C(═O)—R^{48c}, wherein R^{48c} is selected from H and C₁₋₃ alkyl; OH; and R^{49c}—CONH— wherein R^{49c} is selected from C₁₋₃ alkyl and cyclopropyl.

In a still further embodiment C⁶ is selected from a H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen.

In a further embodiment C⁶ is selected from a phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen.

In a still further embodiment C⁶ is selected from Y^{a}—W^{a} wherein
Y^{a} is linked to the 5 membered heterocycle of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

In a still further embodiment B¹ is

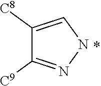

wherein the asterix * indicates the nitrogen atom of the pyrazole ring that is covalently attached to the galactopyranose or cyclohexyl ring.

In an embodiment C⁸ is a phenyl optionally substituted with one, two or three halogens, such as one, two or three F. In another embodiment C⁸ is a heterocycloalkyl, such as morpholinyl, optionally substituted with one or two groups selected from halogen or C₁₋₄ alkyl. Typically, C⁸ is a morpholinyl. In a still other embodiment C⁸ is a C₁₋₆ alkyl, such as C₁₋₄ alkyl, e.g. isopropyl.

In a further embodiment C⁹ is selected from H or C₁₋₆ alkyl, such as H.

In a still further embodiment X¹ is selected from the group consisting of S, Se, SO, SO₂, O, C═O, such as S, SO, or SO₂.

In a further embodiment X¹ is X^{1a}—X^{1b} wherein X^{1a} and X^{1b} are independently selected from the group consisting of S, Se, SO, SO₂, O, C═O. In one embodiment X^{1a} is selected from S, SO, or SO₂, such as S. In a further embodiment X^{1b} is selected from S, SO, or SO₂, such as S. Typically, X¹ is S—S.

In a further embodiment X² is O. Alternatively, X² is CH₂.

In a still further embodiment R¹ is H. In a further embodiment R¹ is OH. In a still further embodiment R¹ is OC₁₋₄ alkyl, such as OCH₃.

In a still further embodiment R² is H. Alternatively, R² is OH.

In a further aspect the present invention concerns a β-D-galactopyranose compound of formula (1) selected from any one of the exemplified compounds of examples 1-14, or a pharmaceutically acceptable salt thereof.

The skilled person will understand that it may be necessary to adjust or change the order of steps in the processes herein, and such change of order is encompassed by the aspects of the process as described above in the reaction schemes and accompanying description of the process steps.

Furthermore, the skilled person will understand that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldipheylsilyl or trimethylsilyl), AcO(acetoxy), TBS(t-butyldimethylsilyl), TMS(trimethylsilyl), PMB (p-methoxybensyl), and tetrahydropyranyl. Suitable protecting groups for carboxylic acid include (C₁₋₆)-alkyl or benzyl esters. Suitable protecting groups for amino include t-butyloxycarbonyl, benzyloxycarbonyl, 2-(trimethylsilyl)-ethoxy-methyl or 2-trimethylsilylethoxycarbonyl (Teoc). Suitable protecting groups for S include S—C(═N)NH₂, TIPS.

The protection and deprotection of functional groups may take place before or after any reaction in the above-mentioned processes.

Furthermore the skilled person will appreciate, that, in order to obtain compounds of the invention in an alternative, and on some occasions more convenient manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

In a still further embodiment the compound (1) is on free form. “On free form” as used herein means a compound of formula (1), either an acid form or base form, or as a neutral compound, depending on the substitutents. The free form does not have any acid salt or base salt in addition. In one embodiment the free form is an anhydrate. In another embodiment the free form is a solvate, such as a hydrate.

In a further embodiment the compound of formula (1) is a crystalline form. The skilled person may carry out tests in order to find polymorphs, and such polymorphs are intended to be encompassed by the term “crystalline form” as used herein.

Whenever a “compound of formula (1)” is used herein it means the compound of formula (1) in any form incl the free form or as a salt thereof, such as a pharmaceutically acceptable salt thereof, unless otherwise indicated herein or clearly contradicted by context.

When the compounds and pharmaceutical compositions herein disclosed are used for the above treatment, a therapeutically effective amount of at least one compound is administered to a mammal in need of said treatment.

The term “C₁₋ₓ alkyl” as used herein means an alkyl group containing 1-x carbon atoms, e.g. C₁₋₅ or C₁₋₆, such as methyl, ethyl, propyl, butyl, pentyl or hexyl.

The term “branched C₃₋₆ alkyl” as used herein means a branched alkyl group containing 3-6 carbon atoms, such as isopropyl, isobutyl, tert-butyl, isopentyl, 3-methylbutyl, 2,2-dimethylpropyl, n-hexyl, 2-methylpentyl, 2,2-dimethylbutyl, 2,3-dimethylbutyl.

The term “C₃₋ₓ cycloalkyl” as used herein means a cyclic alkyl group containing 3-x carbon atoms, e.g. C₃₋₆ or C₃₋₇, such as cyclopropyl, cyclobutyl, cyclopentyl, cyclohexyl, cycloheptyl, and 1-methylcyclopropyl.

The term “C₅₋₇ cycloalkyl” as used herein means a cyclic alkyl group containing 5-7 carbon atoms, such as cyclopentyl, cyclohexyl, or cycloheptyl.

The term “Oxo” as used herein means an oxygen atom with double bonds, also indicated as ═O.

The term “CN” as used herein means a cyano group.

The term “halogen” as used herein means Cl, F, Br or I.

The term “halo-C₁₋₆ alkyl” as used herein means one or more halogens linked to a C₁₋₆ alkyl, such as CF₃, CH(Cl)CHF₂.

The term “C₁₋₆ alkoxy” as used herein means an oxygen linked to a C₁₋₆ alkyl, such as methoxy or ethoxy.

The term “C₁₋₆ alkylthio” as used herein means a sulphur linked to a C₁₋₆ alkyl, such as thiomethoxy or thioethoxy.

The term “halo-C₁₋₆ alkoxy” as used herein means one or more halogens linked to a C₁₋₆ alkoxy, such as CH(F₂)CH(Br)O—.

The term “C₁₋₆ alkoxycarbonyl” as used herein means a C₁₋₆ alkoxy linked to a carbonyl, such as methoxycarbonyl (CH₂OC(═O)).

The term “a five or six membered heteroaromatic ring” as used herein means one five membered heteroaromatic ring or one six membered heteroaromatic ring.

The five membered heteroaromatic ring contains 5 ring atoms of which one to four are heteroatoms selected from N, O, and S. The six membered heteroaromatic ring contains 6 ring atoms of which one to five are heteroatoms selected from N, O and S.

Examples include thiophene, furan, pyran, pyrrole, imidazole, pyrazole, isothiazole, isooxazole, pyridine, pyrazine, pyrimidine and pyridazine. When such heteroaromatic rings are substituents, they are termed thiophenyl, furanyl, pyranyl, pyrrolyl, imidazolyl, pyrazolyl, isothiazolyl, isooxazolyl, pyridinyl, pyrazinyl, pyrimidinyl and pyridazinyl. Also included are oxazoyl, thiazoyl, thiadiazoly, oxadiazoyl, and pyridonyl.

The term “a heterocycle, such as heteroaryl or heterocycloalkyl” as used herein means a heterocycle consisting of one or more 3-7 membered ring systems containing one or more heteroatoms and wherein such ring systems may optionally be aromatic. The term “a heteroaryl” as used herein means a mono or bicyclic aromatic ringsystem containing one or more heteroatoms, such as 1-10, e.g., 1-6, selected from O, S, and N, including but not limited to oxazolyl, oxadiazolyl, thiophenyl, thiadiazolyl, thiazolyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl. The term “a heterocycloalkyl” as used herein means a mono or bicyclic 3-7 membered alifatic heterocycle containing one or more heteroatoms, such as 1-7, e.g., 1-5, selected from O, S, and N, including but not limited to piperidinyl, tetrahydropyranyl, morpholinyl, tetrahydrothipyranyl, or piperidonyl.

The term “a spiro heterocycle” as used herein means a two-ring system connected by a common carbon atom, and containing from 5 to 12 ring members wherein from 2 to 11 are carbon atoms and at least one is a heteroatom, such as a hetero atom selected from one or more N, S, O; one example is N-(2-oxa)-6-azaspiro[3.3]heptanyl.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “a pharmaceutically acceptable salt” as used herein is used to specify that the salt is suitable for use in the human or animal body. An example list of pharmaceutically acceptable salts can be found in the Handbook of Pharmaceutical Salts: Properties, Selection and Use, P. H. Stahl and C. G. Wermuth, editors, Weinheim/Zurich:Wiley-VCH/VHCA, 2002. A pharmaceutically acceptable salt of a compound of Formula (1) includes such salts that may be formed within the human or animal body after administration of said compound to said human or animal body.

The term “a therapeutically effective amount” of a compound of formula (1) of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect, the present invention relates to a pharmaceutical composition comprising the compound of formula (1) and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (1) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person skilled within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99% by weight of said at least one pharmaceutically acceptable adjuvant, diluent, excipient and/or carrier and from 1 to 99% by weight of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compounds as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### Experimental Procedures (Evaluation of Kd Values)

The affinity of Example 1-14 for galectins were determined by a fluorescence anisotropy assay where the compound was used as an inhibitor of the interaction between galectin and a fluorescein tagged saccharide probe as described Sörme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47, (Sarme et al., 2004) and Monovalent interactions of Galectin-1 By Salomonsson, Emma; Larumbe, Amaia; Tejler, Johan; Tullberg, Erik; Rydberg, Hanna; Sundin, Anders; Khabut, Areej; Frejd, Torbjorn; Lobsanov, Yuri D.; Rini, James M.; et al, From Biochemistry (2010), 49(44), 9518-9532, (Salomonsson et al., 2010).

| | | | Galectin- | Galectin- |
|---|---|---|---|---|
| | | | 1 | 3 |
| Example | Name | Structure | Kd (μM) | Kd (μM) |
| 1 | 3,3′-Dideoxy-3-[4- (3,4,5- trifluorophenyl)-1H- 1,2,3-triazol-1-yl]-3′- [4-(2-hydroxythiazol- 4-yl)-1H-1,2,3-triazol- 1-yl]-1,1′-sulfanediyl- di-ß-D- galactopyranoside | 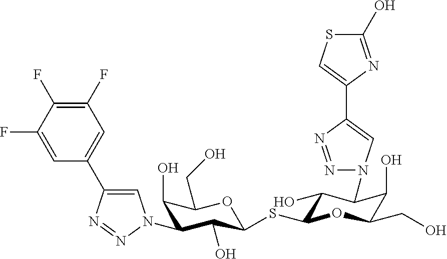 | 0.003 | 0.0004 |
| 2 | 3-[4-(4-Chloro-3,5- difluorophenyl)-1H- 1,2,3-triazol-1-yl]-3,3′- dideoxy-3′-[4-(2- hydroxythiazol-4-yl)- 1H-1,2,3-triazol-1-yl]- 1,1′-sulfanediyl-di-ß- D-galactopyranoside | 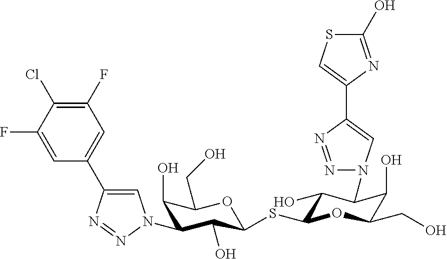 | 0.004 | 0.0003 |
| 3 | 3′-[4-(2-Aminothiazol- 4-yl)-1H-1,2,3-triazol- 1-yl]-3-[4-(4-chloro- 3,5-difluorophenyl)- 1H-1,2,3-triazol-1-yl]- 3,3′-dideoxy-1,1′- sulfanediyl-di-ß-D- galactopyranoside | 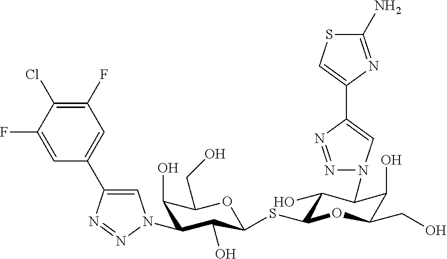 | 0.009 | 0.001 |
| 4 | 1,1′-Sulfanediyl-bis- {3-deoxy-3-[4-(3- fluorophenyl)-1H-1,2- pyrazol-1-yl]-ß-D- galactopyranoside} | 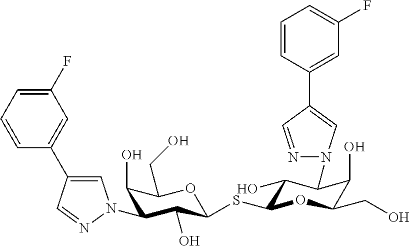 | 0.78 | <0.010 |
| 5 | 1,1′-Sulfanediyl-bis- {3-deoxy-3-[4-(3,4,5- trifluorophenyl)-1H- 1,2-pyrazol-1-yl]-ß-D- galactopyranoside} | 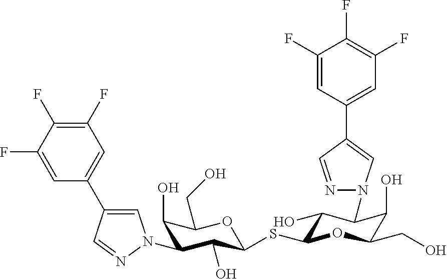 | 0.89 | 0.006 |
| 6 | 3,3′-Dideoxy-3-[4- (3,4,5- trifluorophenyl)-1H- 1,2-pyrazol-1-yl]-3′- [4-(2-hydroxythiazol- 4-yl)-1H-1,2,3-triazol- 1-yl]-1,1′-sulfanediyl- di-ß-D- galactopyranoside | 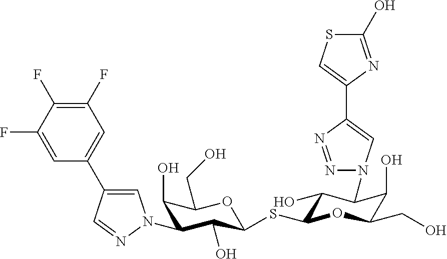 | | |
| 7 | 3,3′-Dideoxy-3-[4- (3,4,5- trifluorophenyl)-1H- 1,2-pyrazol-1-yl]-3′- [4-(morpholin-4-yl)- 1H-1,2-pyrazol-1-yl]- 1,1′-sulfanediyl-di-ß- D-galactopyranoside | 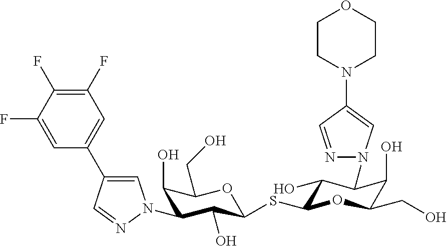 | | |
| 8 | 3,3′-Dideoxy-3-[4- (3,4,5- trifluorophenyl)-1H- 1,2-pyrazol-1-yl]-3′- (4-isopropyl-1H-1,2- pyrazol-1-yl)-1,1′- sulfanediyl-di-ß-D- galactopyranoside | 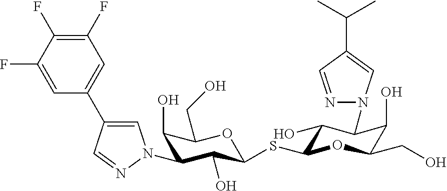 | | |
| 9 | (2S,3R,4S)-3- Hydroxy-4-[4-(3,4,5- trifluorophenyl)-1H- 1,2-pyrazol-1- yl]tetrahydropyran-2- yl 3-deoxy-3-[4-(3,4,5- trifluorophenyl)-1H- 1,2-pyrazol-1-yl]-2-O- methyl-1-thio-ß-D- galactopyranoside | 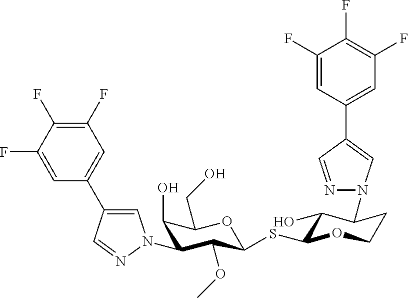 | | |
| 10 | (1R,2R,6S)-1- Hydroxy-6-[4-(3,4,5- trifluorophenyl)-1H- 1,2-pyrazol-1- yl]cyclohexan-2-yl 3- deoxy-3-[4-(3,4,5- trifluorophenyl)-1H- 1,2-pyrazol-1-yl]-2-O- methyl-1-thio-ß-D- galactopyranoside | 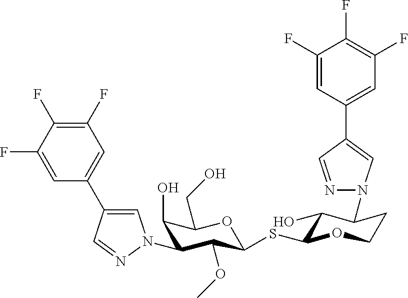 | | |
| 11 | 3-Deoxy-3-[4-(3- fluorophenyl)-1H-1,2- pyrazol-1-yl]-ß-D- galactopyranosyl 3- deoxy-3-[4-(3- fluorophenyl)-1H-1,2- pyrazol-1-yl]-ß-D- galactopyranoside | 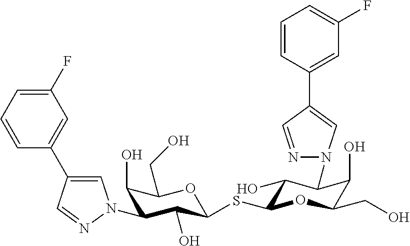 | | |
| 12 | 1,1′-Selenediyl-bis-{3- deoxy-3-[4-(3- fluorophenyl)-1H-1,2- pyrazol-1-yl]-ß-D- galactopyranoside} | 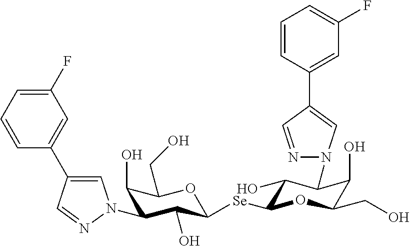 | | |
| 13 | Bis-{3-deoxy-3-[4-(3- fluorophenyl)-1H-1,2- pyrazol-1-yl]-ß-D- galactopyranos-1-yl} disulfide | 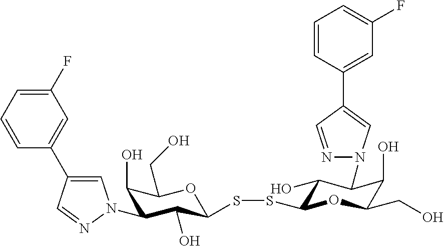 | | |
| 14 | 1,1′-Sulfanediyl-bis- {3-[4-(4-chloro-2,3- difluorophenyl)-1H- 1,2,3-triazol-1-yl]-3- deoxy-ß-D- galactopyranoside} | 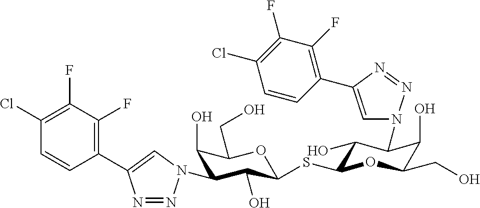 | 0.45 | 0.012 |

### SYNTHESIS OF EXAMPLES AND INTERMEDIATES

### General Experimental:

Nuclear Magnetic Resonance (NMR) spectra were recorded on a 400 MHz Bruker AVANCE III 500 instrument or a Varian instrument at 400 MHz, at 25° C. Chemical shifts are reported in ppm (d) using the residual solvent as internal standard. Peak multiplicities are expressed as follow: s, singlet; d, doublet; dd, doublet of doublets; t, triplet; dt, doublet of triplet; q, quartet; m, multiplet; br s, broad singlet. LC-MS were acquired on an Agilent 1200 HPLC coupled with an Agilent MSD mass spectrometer operating in ES (+) ionization mode. Column: XBridge C18 (4.6×50 mm, 3.5 μm) or SunFire C18 (4.6×50 mm, 3.5 μm). Solvent A water+0.1% TFA and solvent B Acetonitrile+0.1% TFA or solvent A water (10 mM Ammonium hydrogen carbonate) and solvent B Acetonitrile. Wavelength: 254 nM. Alternatively, LC-MS were acquired on an Agilent 1100 HPLC coupled with an Agilent MSD mass spectrometer operating in ES (+) ionization mode. Column: Waters symmetry 2.1×30 mm C18 or Chromolith RP-18 2×50 mm. Solvent A water+0.1% TFA and solvent B Acetonitrile+0.1% TFA. Wavelength 254 nm.

Preparative HPLC was performed on a Gilson 215. Flow: 25 mL/min Column: XBridge prep C18 10 μm OBD (19×250 mm) column. Wavelength: 254 nM. Solvent A water (10 mM Ammonium hydrogen carbonate) and solvent B Acetonitrile. Alternatively, preparative HPLC were acquired on a Gilson system. Flow: 15 ml/min Column: kromasil 100-5-C18 column. Wavelength: 220 nm. Solvent A water+0.1% TFA and solvent B Acetonitrile+0.1% TFA.

The following abbreviations are used
aq: aqueous
Calcd: Calculated
MeCN: Acetonitrile
CuI: Copper Iodide
DCM: Dichloromethane
DIPEA: Diisopropylethylamine
DMF: N,N-dimethylformamide
ESI-MS: Electrospray ionization mass spectrometry
EtOAc or EA: Ethylacetate
Et₃N: Triethylamine
h: hour(s)
HPLC: High performance liquid chromatography
LC: Liquid Chromatography
MeCN: Acetonitrile
mL: milliliter
MeOH: Methanol
MeOD: Deuterated methanol
mm: millimeter
mM: millimolar
MS: Mass spectroscopy
nm: nanometer
NaOMe: Sodium methoxide
N₂: Nitrogen gas
NMR: Nuclear magnetic resonance
PE: petroleum ether
pH: acidity
Prep: preparative
rt: room temperature
TFA: trifluoroacetic acid
THF: Tetrahydrofuran
TMS: Trimethylsilyl
UV: Ultraviolet
Å: Ångström
   Synthesis of examples 1-5 and 14 from their respective intermediates 1-5.
   Synthesis of examples 6-13 are made as described in process steps a1) to a18) above.

### Example 1

### 3,3′-Dideoxy-3-[4-(3,4,5-trifluorophenyl)-1H-1,2,3-triazol-1-yl]-3′-[4-(2-hydroxythiazol-4-yl)-1H-1,2,3-triazol-1-yl]-1,1′-sulfanediyl-di-β-D-galactopyranoside

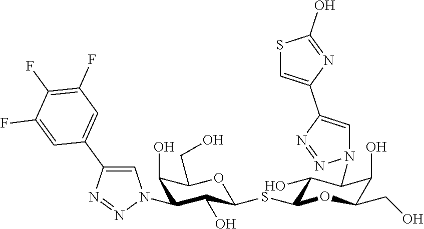

To a solution of 1,1′-sulfanediyl-bis-(2,4,6-tri-O-acetyl-3-azido-3-deoxy-β-D-galactopyranoside) (described in van Scherpenzeel, M; Moret, E. E.; Ballell, L.; Liskamp, R. M. J.; Nilsson, U. J.; Leffler, H.; Pieters, R. J. Synthesis and Evaluation of New Thiodigalactoside-Based Chemical Probes to Label Galectin-3. ChemBioChem, 2009, 10, 1724-1733 as well as in PCT/EP2020/067620 filed 24 Jun. 2020) (400 mg, 0.61 mmol), 4-(2-trimethylsilylethynyl)thiazol-2-ol (120 mg, 0.61 mmol) and CuI (24 mg, 0.12 mmol) in MeCN (4 mL) DIPEA (0.14 mL, 0.78 mmol) followed by TBAF (61 μL, 1 M in THF, 0.061 mmol) were added and the mixture was stirred 6 h at 50° C. The mixture was purified by chromatography (SiO₂, EtOAc). The obtained material (358 mg) was dissolved together with 3,4,5-trifluorophenylacetylene (156 mg, 0.68 mmol) and CuI (18 mg, 0.09 mmol) in MeCN (5 mL). To the solution DIPEA (0.10 mL, 0.59 mmol) followed by TBAF (50 μL, 1 M in THF, 0.050 mmol) were added and the mixture was stirred 6 h at 50° C. The mixture was purified by chromatography (SiO₂, EtOAc). The obtained material (81 mg) was stirred 90 min at rt in MeOH (1.5 mL) and NaOMe (0.25 mL, 1 M). The mixture was purified by chromatography (SiO₂, EtOAc/MeOH) and further purification by prep HPLC (C₁₈, H₂O/MeCN/0.1% TFA) to afford the title compound (40 mg, 10%). ESI-MS m/z calcd for [C₂₅H₂₆F₃N₇O₉S₂] [M+H]⁺: 690.1; found: 690.2. ¹H NMR (400 MHz, Methanol-d₄) δ 8.56 (s, 1H), 8.40 (s, 1H), 7.66-7.56 (m, 2H), 6.63 (s, 1H), 4.97-4.86 (m, 4H), 4.60 (t, J=10.1 Hz, 1H), 4.55 (t, J=10.1 Hz, 1H), 4.14 (s, 2H), 3.91-3.78 (m, 4H), 3.71 (dd, J=11.2, 4.4 Hz, 2H).

### Example 2

### 3-[4-(4-Chloro-3,5-difluorophenyl)-1H-1,2,3-triazol-1-yl]-3,3′-dideoxy-3′-[4-(2-hydroxythiazol-4-yl)-1H-1,2,3-triazol-1-yl]-1,1′-sulfanediyl-di-β-D-galactopyranoside

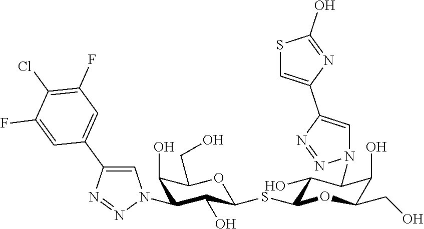

To a solution of 1,1′-sulfanediyl-bis-(2,4,6-tri-O-acetyl-3-azido-3-deoxy-β-D-galactopyranoside) (400 mg, 0.61 mmol), 4-(2-trimethylsilylethynyl)thiazol-2-ol (120 mg, 0.61 mmol), 4-chloro-3,5-difluorophenylacetylene (150 mg, 0.61 mmol) and CuI (24 mg, 0.12 mmol) in MeCN (6 mL) DIPEA (0.14 mL, 0.78 mmol) followed by TBAF (61 μL, 1 M in THF, 0.061 mmol) were added and the mixture was stirred 3 h at 50° C. The mixture was partitioned between EtOAc, water and HCl (2 mL, 1 M), and the organic phase was dried and evaporated. The residue was first purified by prep HPLC (C₁₈, H₂O/MeCN/0.1% TFA), then by chromatography (SiO₂, PE/EtOAc). The obtained material (64 mg) was stirred 30 min at rt in MeOH (3.0 mL) and NaOMe (0.5 mL, 1 M). The mixture was purified by chromatography (SiO₂, EtOAc/MeOH) to afford the title compound (43 mg, 10%). ESI-MS m/z calcd for [C₂₅H₂₆ClF₂N₇O₉S₂] [M+H]⁺: 706.1; found: 706.2. ¹H NMR (400 MHz, Methanol-d₄) δ 8.61 (s, 1H), 8.41 (s, 1H), 7.62 (d, J=7.9 Hz, 2H), 6.64 (s, 1H), 4.97-4.88 (m, 4H), 4.61 (t, J=10.1 Hz, 1H), 4.56 (t, J=10.1 Hz, 1H), 4.15 (s, 2H), 3.92-3.78 (m, 4H), 3.72 (dd, J=11.3, 4.5 Hz, 2H).

### Example 3

### 3′-[4-(2-Aminothiazol-4-yl)-1H-1,2,3-triazol-1-yl]-3-[4-(4-chloro-3,5-difluorophenyl)-1H-1,2,3-triazol-1-yl]-3,3′-dideoxy-1,1′-sulfanediyl-di-β-D-galactopyranoside

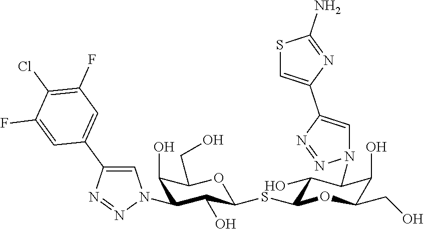

To a solution of 3′-azido-3-[4-(4-chloro-3,5-difluorophenyl)-1H-1,2,3-triazol-1-yl]-3,3′-dideoxy-1,1′-sulfanediyl-di-β-D-galactopyranoside (32 mg, 0.055 mmol), 4-(2-trimethylsilylethynyl)thiazol-2-amine (16 mg, 0.083 mmol) and CuI (1.1 mg, 0.0055 mmol) in MeCN (0.5 mL) DIPEA (19 μL, 0.78 mmol) was added and the mixture was stirred 42 h at 50° C. The mixture was purified by prep HPLC (C₁₈, H₂O/MeCN/0.1% TFA) to afford the title compound (22 mg, 57%). ESI-MS m/z calcd for [C₂₅H₂₇ClF₂N₈O₈S₂] [M+H]⁺: 705.1; found: 705.2. ¹H NMR (400 MHz, Methanol-d₄) δ 8.62 (s, 1H), 8.53 (s, 1H), 7.61 (d, J=7.9 Hz, 2H), 7.06 (s, 1H), 4.97-4.89 (m, 4H), 4.67-4.57 (m, 2H), 4.17 (d, J=2.4 Hz, 2H), 3.92-3.79 (m, 4H), 3.72 (dt, J=11.4, 3.7 Hz, 2H).

### Example 4

### 1,1′-Sulfanediyl-bis-{3-deoxy-3-[4-(3-fluorophenyl)-1H-1,2-pyrazol-1-yl]-β-D-galactopyranoside}

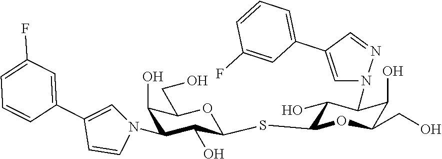

To a solution of 1,1′-sulfanediyl-bis-{4,6-O-benzylidene-3-[(2-tert-butoxycarbonyl)hydrazinyl]-3-deoxy-β-D-galactopyranoside} (85 mg, 0.11 mmol) in EtOH (4 mL) 3-(dimethylamino)-2-(3-fluorophenyl)prop-2-enal (47 mg, 0.25 mmol) and concentrated HCl (0.2 mL) were added and the mixture was stirred 2 h at 80° C. The mixture was evaporated and purified by preparative HPLC (MeCN/H₂O (10 mmol/L NH₄HCO₃), X-Select 10 μm 19*250 mm, 20 mL/min, UV 254) to afford the title compound (6.88 mg, 10%). ESI-MS m/z calcd for [C₃₀H₃₂F₂N₄O₈S] [M+H]⁺: 647.2; found: 648.8. ¹H NMR (400 MHz, Methanol-d₄) δ 8.25 (s, 2H), 7.90 (s, 2H), 7.42-7.27 (m, 6H), 6.93-6.87 (m, 2H), 4.85 (d, J=9.6 Hz, 2H), 4.66 (t, J=10.0 Hz, 2H), 4.41 (dd, J=10.8, 2.8 Hz, 2H), 4.12 (d, J=2.8 Hz, 2H), 3.87-3.77 (m, 4H), 3.73-3.68 (m, 2H).

### Example 5

### 1,1′-Sulfanediyl-bis-{3-deoxy-3-[4-(3,4,5-trifluorophenyl)-1H-1,2-pyrazol-1-yl]-β-D-galactopyranoside}

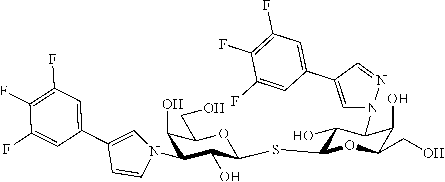

To a solution of 1,1′-sulfanediyl-bis-{4,6-O-benzylidene-3-[(2-tert-butoxycarbonyl)hydrazinyl]-3-deoxy-β-D-galactopyranoside} (55 mg, 0.072 mmol) in DCM (2 mL) TFA (0.2 mL) was added and the mixture was stirred overnight at rt. The mixture was evaporated and dissolved in EtOH (4 mL). 3-(Dimethylamino)-2-(3,4,5-trifluorophenyl)prop-2-enal (49.6 mg, 0.22 mmol) and concentrated HCl (0.2 mL) were added and the mixture was stirred 2 h at 80° C. The mixture was evaporated and purified by preparative HPLC (MeCN/H₂O (10 mmol/L NH₄HCO₃), X-Select 10 μm 19*250 mm, 20 mL/min, UV 254) to afford the title compound (8.08 mg, 16%). ESI-MS m/z calcd for [C₃₀H₂₈F₆N₄O₈S] [M+H]⁺: 719.2; found: 718.8. ¹H NMR (400 MHz, Methanol-d₄) δ 8.25 (s, 2H), 7.90 (s, 2H), 7.53-7.20 (m, 4H), 4.85 (d, J=9.6 Hz, 2H), 4.64 (t, J=10.1 Hz, 2H), 4.41 (dd, J=10.4, 2.8 Hz, 2H), 4.11 (d, J=2.8 Hz, 2H), 3.87-3.77 (m, 4H), 3.71 (dd, J=16, 8.8 Hz, 2H).

### Examples 6-13 are Made as Described in Process Steps a1) to a18) Above

### Example 14

### 1,1′-Sulfanediyl-bis-{3-[4-(4-chloro-2,3-difluorophenyl)-1H-1,2,3-triazol-1-yl]-3-deoxy-β-D-galactopyranoside}

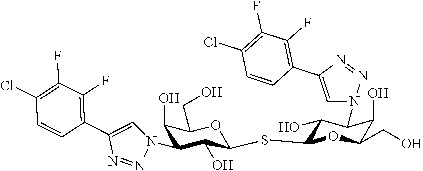

To a solution of 1,1′-sulfanediyl-bis-(2,4,6-tri-O-acetyl-3-azido-3-deoxy-β-D-galactopyranoside) (100 mg, 0.15 mmol) and 2-(4-chloro-2,3-difluorophenyl)ethynyltrimethylsilane (77.8 mg, 0.32 mmol) and CuI (2.9 mg, 0.12 mmol) in MeCN (5 mL) DIPEA (0.079 mL, 0.45 mmol) followed by TBAF (15 μL, 1 M in THF, 0.015 mmol) were added and the mixture was stirred overnight at 50° C. Additional CuI (2.9 mg, 0.12 mmol) and TBAF (15 μL, 1 M in THF, 0.015 mmol) were added and the mixture was stirred 3 h at 50° C. The mixture was diluted with MeOH and water and purified by prep HPLC (C₁₈, H₂O/MeCN/0.1% TFA). The obtained material was stirred overnight at 35° C. in MeOH (5 mL) and NaOMe (60.6 μL, 5 M in THF, 0.30 mmol). The mixture was purified by prep HPLC (C₁₈, H₂O/MeCN/0.1% TFA) to afford the title compound (13 mg, 11%). ESI-MS m/z calcd for [C₂₈H₂₆Cl₂F₄N₆O₈S] [M+H]⁺: 753.1; found: 753.0. ¹H NMR (400 MHz, Methanol-d₄) δ 8.56 (d, J=3.3 Hz, 2H), 7.90 (s, 2H), 7.40 (s, 2H), 4.99-4.81 (m, 6H), 4.16 (d, J=2.7 Hz, 2H), 3.93-3.81 (m, 4H), 3.72 (dd, J=10.4, 3.0 Hz, 2H).

### Intermediate 3

### 3′-Azido-3-[4-(4-chloro-3,5-difluorophenyl)-1H-1,2,3-triazol-1-yl]-3,3′-dideoxy-1,1′-sulfanediyl-di-β-D-galactopyranoside

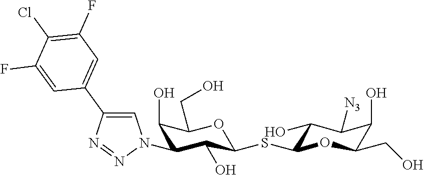

To a solution of 1,1′-sulfanediyl-bis-(2,4,6-tri-O-acetyl-3-azido-3-deoxy-β-D-galactopyranoside) (1.00 g, 1.51 mmol), 4-chloro-3,5-difluorophenylacetylene (370 mg, 1.51 mmol) and CuI (29 mg, 0.15 mmol) in MeCN (10 mL) DIPEA (0.52 mL, 3.0 mmol) was added and the mixture was stirred 3 h at 50° C. The mixture was partitioned between EtOAc and brine. The organic phase was dried, evaporated, and purified by chromatography (SiO₂, PE/EtOAc). The obtained material (700 mg) was stirred 14 h at rt in MeOH (21 mL) and NaOMe (7 mL, 1 M). The mixture was neutralized with acetic acid, evaporated, and purified by HPLC (C₁₈, H₂O/MeCN/0.1% TFA) to afford the product (204 mg, 23%). ESI-MS m/z calcd for [C₂₀H₂₃ClF₂N₆O₈S] [M+H]⁺: 581.1; found: 581.2. ¹H NMR (400 MHz, Methanol-d₄) δ 8.58 (s, 1H), 7.61 (d, J=8.1 Hz, 2H), 4.95-4.88 (m, 2H), 4.77 (d, J=9.7 Hz, 1H), 4.44 (t, J=10.1 Hz, 1H), 4.14 (d, J=3.0 Hz, 1H), 4.04-3.97 (m, 2H), 3.90-3.58 (m, 6H), 3.39 (dd, J=9.9, 3.0 Hz, 1H).

### Intermediate 4

### 3-(Dimethylamino)-2-(3-fluorophenyl)prop-2-enal

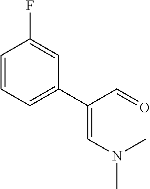

To a cooled (0° C.) solution of N,N-dimethylformamide (2134 mg, 29.2 mmol) and phosphoryl trichloride (4029 mg, 26.3 mmol) 2-(3-fluorophenyl)acetic acid (900 mg, 5.84 mmol) was added and the mixture was stirred overnight at 60-70° C. After cooling to rt, the mixture was slowly added to a mixture of ice and water with external cooling and ice intermittently to keep the temperature <10° C. K₂CO₃ was added slowly until pH=11 was achieved. Small quantities of ethanol (2 mL) were added to control frothing. To the alkaline mixture toluene (30 mL) was added, and the mixture was refluxed for 1.5 h and cooled to rt. The aqueous layer was extracted with an additional 40 mL of toluene. The combined organic layers were washed with water, dried using Na₂SO₄ and evaporated. The obtained solid was recrystallized from hexane to give the product (560 mg, 50%). ESI-MS m/z calcd for [C₁₁H₁₂FNO] [M+H]⁺: 194.1; found: 194.2. ¹H NMR (400 MHz, Chloroform-d) δ 9.02 (s, 1H), 7.26-7.21 (m, 1H), 6.96-6.66 (m, 4H), 2.80 (s, 6H).

### 1,1′-Sulfanediyl-bis-(3-azido-3-deoxy-β-D-galactopyranoside)

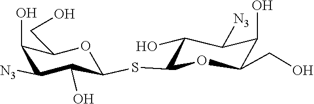

To a solution of 1,1′-sulfanediyl-bis-(2,4,6-tri-O-acetyl-3-azido-3-deoxy-β-D-galactopyranoside) (800 mg, 1.21 mmol) in MeOH (10 mL) NaOMe (65.4 mg, 1.21 mmol) was added and the mixture was stirred 2 h at rt. The mixture was evaporated and purified by chromatography (MeOH/DCM=0/1˜1/3, Silica-CS 20 g, 25 mL/min, silica gel, UV 254) to afford the product (280 mg, 57%). ¹H NMR (400 MHz, Methanol-d₄) δ 4.71 (d, J=9.6 Hz, 2H), 3.93 (d, J=2.4 Hz, 2H), 3.83 (t, J=9.8 Hz, 2H), 3.73 (dd, J=11.6, 7.2 Hz, 2H), 3.63 (dd, J=11.6, 4.8 Hz, 2H), 3.58-3.53 (m, 2H), 3.34 (dd, J=10.0, 2.8 Hz, 2H).

### 1,1′-Sulfanediyl-bis-(3-azido-4,6-O-benzylidene-3-deoxy-β-D-galactopyranoside)

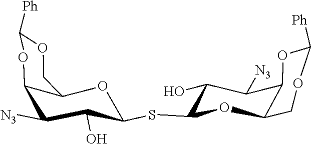

To a solution of 1,1′-sulfanediyl-bis-(3-azido-3-deoxy-β-D-galactopyranoside) (410 mg, 1.00 mmol) in DMF (3 mL) benzaldehyde dimethylacetal (489 mg, 3.21 mmol) and D(+)-10-camphorsulfonic acid (70 mg, 0.30 mmol) were added and the mixture was stirred 3 h at 60° C. The mixture was evaporated and purified by chromatography (DCM/EtOAc=10/1˜3/2, Silica-CS 12 g, 20 mL/min, silica gel, UV 254) to afford the product (300 mg, 51%). ESI-MS m/z calcd for [C₂₆H₂₈N₆O₈S] [M+NH₄]⁺: 602.2; found: 602.2. ¹H NMR (400 MHz, Chloroform-d) δ 7.50-7.39 (m, 4H), 7.25-7.16 (m, 6H), 5.51 (s, 2H), 4.55 (td, J=9.6, 3.6 Hz, 2H), 4.44 (d, J=9.6 Hz, 2H), 4.31 (dd, J=12.6, 1.4 Hz, 2H), 4.21 (d, J=3.2 Hz, 2H), 4.03 (dd, J=12.8, 1.6 Hz, 2H), 3.58-3.49 (m, 4H), 3.42 (d, J=3.6 Hz, 2H).

### 1,1′-Sulfanediyl-bis-(3-amino-4,6-O-benzylidene-3-deoxy-β-D-galactopyranoside)

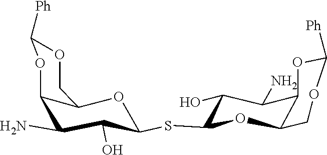

To a solution of 1,1′-sulfanediyl-bis-(3-azido-4,6-O-benzylidene-3-deoxy-β-D-galactopyranoside) (300 mg, 0.51 mmol) in THF (10 mL) and water (2 mL) triphenylphosphine (1.01 g, 4.11 mmol) was added and the mixture was stirred overnight at 50° C. The was evaporated and purified by column chromatography (MeOH/DCM=0/1˜2/3, Silica-CS 12 g, 20 m/min, silica gel, UV 254) to the product (220 mg, 81%). ESI-MS m/z calcd for [C₂₆H₃₂N₂O₈S] [M+H]⁺: 533.2; found: 533.0. ¹H NMR (400 MHz, Chloroform-d) δ 7.41-7.35 (m, 4H), 7.33-7.23 (m, 6H), 5.51 (s, 2H), 4.31 (d, J=9.6 Hz, 2H), 4.27 (d, J=12.4, 2H), 4.16 (t, J=10.2 Hz, 2H), 4.13-4.09 (m, 2H), 4.02 (d, J=11.6 Hz, 2H), 3.51 (s, 2H), 2.64 (dd, J=9.6, 2.8 Hz, 2H).

### 1,1′-Sulfanediyl-bis-{4,6-O-benzylidene-3-[(2-tert-butoxycarbonyl)hydrazinyl]-3-deoxy-β-D-galactopyranoside}

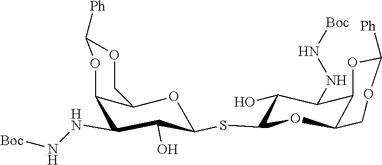

To a solution of 1,1′-sulfanediyl-bis-(3-amino-4,6-O-benzylidene-3-deoxy-β-D-galactopyranoside) (220 mg, 0.41 mmol) in DCM (5 mL) tert-butyl 3-(4-cyanophenyl)oxaziridine-2-carboxylate (224 mg, 0.91 mmol) was added and the mixture was stirred overnight at rt. The was evaporated and purified by column chromatography (MeOH/DCM=0/11˜/5, Silica-CS 12 g, 20 m/min, silica gel, UV 254) to give the product (180 mg, 57%). ESI-MS m/z calcd for [C₃₆H₅₀N₄O₁₂S] [M+H]⁺: 763.3; found: 763.0. ¹H NMR (400 MHz, Chloroform-d) δ 7.41-7.09 (m, 10H), 6.60 (s, 2H), 5.48 (s, 2H), 4.48-4.45 (m, 2H), 4.38-4.31 (m, 4H), 4.27 (d, J=12.4 Hz, 2H), 4.02 (d, J=11.6 Hz, 2H), 3.55-3.45 (m, 6H), 2.86 (t, J=8.0 Hz, 2H), 1.47 (s, 18H).

### Intermediate 5

### 3-(Dimethylamino)-2-(3,4,5-trifluorophenyl)prop-2-enal

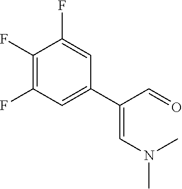 [SMILES: CN(C)/C=C(\C=O)c1cc(F)c(F)c(F)c1]

To a cooled (0° C.) solution of N,N-dimethylformamide (913 mg, 12.5 mmol) and phosphoryl trichloride (1724 mg, 11.2 mmol) 2-(3,4,5-trifluorophenyl)acetic acid (475 mg, 2.50 mmol) was added and the mixture was stirred overnight at 60-70° C. After cooling to rt, the mixture was slowly added to a mixture of ice and water with external cooling and ice intermittently to keep the temperature <10° C. K₂CO₃ was added slowly until pH=11 was achieved. Small quantities of ethanol were added to control frothing. To the alkaline mixture toluene (10 mL) was added, and the mixture was refluxed for 1.5 h and cooled to rt. The aqueous layer was extracted with an additional 20 mL of toluene. The combined organic layers were washed with water, dried using Na₂SO₄ and evaporated. The obtained solid was recrystallized from hexane to give the product (320 mg, 56%). ESI-MS m/z calcd for [C₁₁H₁₀F₃NO] [M+H]⁺: 230.1; found: 230.1. ¹H NMR (400 MHz, Chloroform-d) δ 9.05 (s, 1H), 6.91 (s, 1H), 6.82 (dd, J=8.2, 6.6 Hz, 2H), 2.91 (s, 6H).

### REFERENCES

Bennett, D.; Bargagli, E.; Bianchi, N.; Landi, C.; Fossi, A.; Fui, A.; Sestini, P.; Refini, R. M.; Rottoli, P. Elevated Level of Galectin-1 in Bronchoalveolar Lavage of Patients with Idiopathic Pulmonary Fibrosis. Respir Physiol Neurobiol 2019, 273, 103323. https://doi.org/10.1016/j.resp.2019.103323.
Blanchard, H.; Yu, X.; Collins, P. M.; Bum-Erdene, K. Galectin-3 Inhibitors: A Patent Review (2008-Present). Expert Opin. Ther. Patents 2014, 24 (10), 1053-1065. https://doi.org/10.1517/13543776.2014.947961.
Blanchard, H.; Bum-Erdene, K.; Bohari, M. H.; Yu, X. Galectin-1 Inhibitors and Their Potential Therapeutic Applications: A Patent Review. Expert Opin. Ther. Patents 2016, 26 (5), 537-554. https://doi.org/10.1517/13543776.2016.1163338.
Daley, D.; Mani, V. R.; Mohan, N.; Akkad, N.; Ochi, A.; Heindel, D. W.; Lee, K. B.; Zambirinis, C. P.; Pandian, G. S. D. B.; Savadkar, S.; Torres-Hernandez, A.; Nayak, S.; Wang, D.; Hundeyin, M.; Diskin, B.; Aykut, B.; Werba, G.; Barilla, R. M.; Rodriguez, R.; Chang, S.; Gardner, L.; Mahal, L. K.; Ueberheide, B.; Miller, G. Dectin-1 Activation on Macrophages by Galectin-9 Promotes Pancreatic Carcinoma and Peritumoral Immune-Tolerance. Nat Med 2017, 23 (5), 556-567. https://doi.org/10.1038/nm.4314.
Dings, R. P. M., Miller, M. C., Griffin, R. J. & Mayo, K. H. Galectins as Molecular Targets for Therapeutic Intervention. IJMS 19, (2018).
Dubé-Delarosbil, C.; St-Pierre, Y. The Emerging Role of Galectins in High-Fatality Cancers. Cell. Mol. Life Sci. 2017, 75 (7), 1215-1226. https://doi.org/10.1007/s00018-017-2708-5.
Drake, I.; Fryk, E.; Strindberg, L.; Lundqvist, A.; Rosengren, A. H.; Groop, L.; Ahlqvist, E.; Bordn, J.; Orho-Melander, M.; Jansson, P.-A. The Role of Circulating Galectin-1 in Type 2 Diabetes and Chronic Kidney Disease: Evidence from Cross-Sectional, Longitudinal and Mendelian Randomisation Analyses. Diabetologia 2022, 65 (1), 128-139. https://doi.org/10.1007/s00125-021-05594-1.
Hsu, Y.-A.; Chang, C.-Y.; Lan, J.-L.; Li, J.-P.; Lin, H.-J.; Chen, C.-S.; Wan, L.; Liu, F.-T. Amelioration of Bleomycin-Induced Pulmonary Fibrosis via TGF-β-Induced Smad and Non-Smad Signaling Pathways in Galectin-9-Deficient Mice and Fibroblast Cells. J Biomed Sci 2020, 27 (1), 24. https://doi.org/10.1186/s12929-020-0616-8.
Johannes, L.; Jacob, R.; Leffler, H. Galectins at a Glance. J Cell Sci 2018, 131 (9), jcs208884. https://doi.org/10.1242/jcs.208884.
Kathiriya, J. J.; Nakra, N.; Nixon, J.; Patel, P. S.; Vaghasiya, V.; Alhassani, A.; Tian, Z.; Allen-Gipson, D.; Dav, V. Galectin-1 Inhibition Attenuates Profibrotic Signaling in Hypoxia-Induced Pulmonary Fibrosis. Cell Death Discovery 2017, 3, 17010-17013. https://doi.org/10.1038/cddiscovery.2017.10.
Li, P.; Liu, S.; Lu, M.; Bandyopadhyay, G.; Oh, D.; Imamura, T.; Johnson, A. M. F.; Sears, D.; Shen, Z.; Cui, B.; Kong, L.; Hou, S.; Liang, X.; Iovino, S.; Watkins, S. M.; Ying, W.; Osborn, O.; Wollam, J.; Brenner, M.; Olefsky, J. M. Hematopoietic-Derived Galectin-3 Causes Cellular and Systemic Insulin Resistance. Cell 2016, 167 (4), 973-984.e12. https://doi.org/10.1016/j.cell.2016.10.025.
Sethi, A.; Sanam, S.; Alvala, R.; Alvala, M. An Updated Patent Review of Galectin-1 and Galectin-3 Inhibitors and Their Potential Therapeutic Applications (2016-Present). Expert Opin Ther Pat 2021, 31 (8), 1-13. https://doi.org/10.1080/13543776.2021.1903430.
Slack, R. J.; Mills, R.; Mackinnon, A. C. The Therapeutic Potential of Galectin-3 Inhibition in Fibrotic Disease. Int J Biochem Cell Biology 2020, 105881. https://doi.org/10.1016/j.biocel.2020.105881.
Sundblad, V., Morosi, L. G., Geffner, J. R. & Rabinovich, G. A. Galectin-1: A Jack-of-All-Trades in the Resolution of Acute and Chronic Inflammation. J. Immunol. 199, 3721-3730 (2017).
Wolf, Y.; Anderson, A. C.; Kuchroo, V. K. TIM3 Comes of Age as an Inhibitory Receptor. Nat Rev Immunol 2020, 20 (3), 173-185. https://doi.org/10.1038/s41577-019-0224-6.
Wu, D.; Kanda, A.; Liu, Y.; Kase, S.; Noda, K.; Ishida, S. Galectin-1 Promotes Choroidal Neovascularization and Subretinal Fibrosis Mediated via Epithelial-Mesenchymal Transition. FASEB J. 2019, 33 (2), 2498-2513. https://doi.org/10.1096/fj.201801227r.
Yang, R.; Sun, L.; Li, C.-F.; Wang, Y.-H.; Yao, J.; Li, H.; Yan, M.; Chang, W.-C.; Hsu, J.-M.; Cha, J.-H.; Hsu, J. L.; Chou, C.-W.; Sun, X.; Deng, Y.; Chou, C.-K.; Yu, D.; Hung, M.-C. Galectin-9 Interacts with PD-1 and TIM-3 to Regulate T Cell Death and Is a Target for Cancer Immunotherapy. Nat Commun 2021, 12 (1), 832. https://doi.org/10.1038/s41467-021-21099-2.

1-15. (canceled)

16. A D-galactopyranose compound of formula (1) 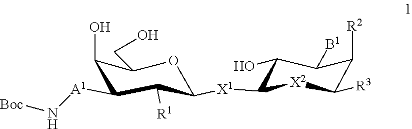
wherein
the pyranose ring whereto A¹ and R¹ is attached is β-D-galactopyranose,
A¹ is (R⁴)ₙ—Z¹,
wherein
Z¹ is a five membered heterocycle having at least one heteroatom selected from 0, S, and N and is attached to the C3 position of the β-D-galactopyranose;
n is 1 or 2,
each R⁴ is independently selected from
a) an aryl, optionally substituted with a group selected from a halogen; CN; a spiro heterocycle; —COOH; —CONR⁵R⁶, wherein R⁵ and R⁶ are independently selected from H, C₁₋₃ alkyl, and cyclopropyl, or R⁵ and R⁶ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁷R⁸, wherein R⁷ and R⁸ are independently selected from H, C₁₋₃ alkyl and cyclopropyl; C(═O)—R⁹, wherein R⁹ is selected from H and C₁₋₃ alkyl OH; and R¹⁰—CONH— wherein R¹⁰ is selected from C₁₋₃ alkyl and cyclopropyl;
b) a heterocycle, optionally substituted with a group selected from a halogen; a spiro heterocycle; CN; —COOH; —CONR¹¹R¹², wherein R¹¹ and R¹² are independently selected from H, C₁₋₃ alkyl, and cyclopropyl, or R¹¹ and R¹² together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR¹³R¹⁴, wherein R¹³ and R¹⁴ are independently selected from H, C₁₋₃ alkyl, and cyclopropyl; C(═O)—R¹⁵, wherein R¹⁵ is selected from H and C₁₋₃ alkyl; OH; and R¹⁶—CONH— wherein R¹⁶ is selected from C₁₋₃ alkyl and cyclopropyl;
c) a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen;
d) a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen;
e) Y—W wherein
Y is linked to the 5 membered heterocycle Z¹ of A¹ and is selected from the group consisting of S, Se, SO, SO₂, O, C═O, and CR⁶²R⁶³ wherein R⁶² and R⁶³ are independently selected from hydrogen, OH, or halogen; and
W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen;
B¹ is (R^{4a})ₘ—Z²,
Wherein
Z² is a five membered heterocycle having at least one heteroatom selected from O, S, and N and is attached to the C3 position of the D-galactopyranose or the C3 position of the cyclohexyl ring;
m is 1 or 2,
each R^{4a} is independently selected from
a) an aryl, optionally substituted with a group selected from a halogen; CN; a spiro heterocycle; —COOH; —CONR^{5a}R^{6a}, wherein R^{5a} and R^{6a} are independently selected from H, C₁₋₃ alkyl, and cyclopropyl, or R^{5a} and R^{6a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{7a}R^{8a}, wherein R^{7a} and R^{8a} are independently selected from H, C₁₋₃ alkyl and cyclopropyl; C(═O)—R^{9a}, wherein R^{9a} is selected from H and C₁₋₃ alkyl OH; and R^{10a}—CONH— wherein R^{10a} is selected from C₁₋₃ alkyl and cyclopropyl;
b) a heterocycle, optionally substituted with a group selected from a halogen; a spiro heterocycle; CN; —COOH; —CONR^{11a}R^{12a}, wherein R^{11a} and R^{12a} are independently selected from H, C₁₋₃ alkyl, and cyclopropyl, or R^{11a} and R^{12a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{13a}R^{14a}, wherein R^{13a} and R^{14a} are independently selected from H, C₁₋₃ alkyl, and cyclopropyl; C(═O)—R^{5a}, wherein R^{15a} is selected from H and C₁₋₃ alkyl; OH; and R^{16a}—CONH— wherein R^{16a} is selected from C₁₋₃ alkyl and cyclopropyl;
c) a group selected from H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R^{50a} and R^{51a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen;
d) a group selected from phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen;
e) Y^{a}—W^{a} wherein
Y^{a} is linked to the 5 membered heterocycle Z² of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen;
X¹ is selected from the group consisting of S, Se, SO, SO₂, O, C═O, and CR¹⁷R¹⁸ wherein R¹⁷ and R¹⁵ are independently selected from hydrogen, OH, or halogen, or X¹ is X^{1a}—X^{1b} wherein X^{1a} and X^{1b} are independently selected from the group consisting of S, Se, SO, SO₂, O, C═O, and CR¹⁹R²⁰ wherein R¹⁹ and R²⁰ are independently selected from hydrogen, OH, or halogen;
X² is O or CH₂;
R¹ is selected from the group consisting of a) H, b) OH, c) OC₁₋₆ alkyl optionally substituted with one or more halogen, phenyl, phenyl substituted with one or more groups selected form OH and halogen, CN, OR²¹, NR²²R²³, and CONH₂, wherein R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, and R²³ is selected from the group consisting of H, CN, a halogen, methyl optionally substituted with a F, OCH₃ optionally substituted with a F, OCH₂CH₃ optionally substituted with a F, OH, and R²⁶—CONH— wherein R²⁶ is selected from C₁₋₃ alkyl and cyclopropyl, d) branched OC₃₋₆ alkyl optionally substituted with one or more halogen, CN, OR²⁷, NR²⁸R²⁹, and CONH₂, wherein R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R³⁰ is selected from C₁₋₃ alkyl and cyclopropyl, R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, and R²⁹ is selected from the group consisting of H, CN, a halogen, methyl optionally substituted with a F, OCH₃ optionally substituted with a F, OCH₂CH₃ optionally substituted with a F, OH, and R³²—CONH— wherein R³² is selected from C₁₋₃ alkyl and cyclopropyl, e) cyclic OC₃₋₆ alkyl optionally substituted with one or more halogen, CN, OR³³, NR³⁴R³⁵, and CONH₂, wherein R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and R³⁵ is selected from the group consisting of H, CN, a halogen, methyl optionally substituted with a F, OCH₃ optionally substituted with a F, OCH₂CH₃ optionally substituted with a F, OH, and R³⁸—CONH— wherein R³⁸ is selected from C₁₋₃ alkyl and cyclopropyl, and f) a fluorine (F);
R² is selected from H or OH
R³ is selected from H or CH₂OH; or
a pharmaceutically acceptable salt or solvate thereof.

17. The compound of claim 16 wherein A¹ is 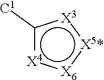
wherein the asterix * indicates the X⁵ atom of the five membered heterocycle that is covalently attached to the galactopyranose,
X³, X⁴, and X⁶ are independently selected from CH, N, O and S, provided that the five membered heterocycle is aromatic,
X⁵ is carbon or nitrogen,
C¹ is a) a phenyl, optionally substituted with a group selected from a halogen; CN; —COOH; —CONR³⁹R⁴⁰, wherein R³⁹ and R⁴⁰ are independently selected from H, C₁₋₃ alkyl and cyclopropyl, or R³⁹ and R⁴⁰ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴¹R⁴², wherein R⁴¹ and R⁴² are independently selected from H and C₁₋₃ alkyl; OH; and R⁴³—CONH— wherein R⁴³ is selected from C₁₋₃ alkyl and cyclopropyl; or b) a heterocycle, optionally substituted with a group selected from a halogen; a spiro heterocycle; CN; —COOH; —CONR⁴⁴R⁴⁵, wherein R⁴⁴ and R⁴⁵ are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴⁶R⁴⁷, wherein R⁴⁶ and R⁴⁷ are independently selected from H, C₁₋₃ alkyl and cyclopropyl, C(═O)—R⁴⁸, wherein R⁴⁸ is selected from H and C₁₋₃ alkyl; OH; and R⁴⁹—CONH— wherein R⁴⁹ is selected from C₁₋₃ alkyl and cyclopropyl.

18. The compound of claim 17 wherein A¹ is 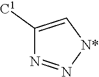
wherein the asterix * indicates the nitrogen atom of the triazole ring that is covalently attached to the galactopyranose; and
C¹ is as defined in claim 17.

19. The compound of claim 16 wherein A¹ is 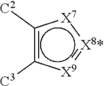
wherein the asterix * indicates the X⁸ atom of the five membered heterocycle that is covalently attached to the galactopyranose,
X⁷ and X⁹ are independently selected from CH, N, O and S, provided that the five membered heterocycle is aromatic,
X⁸ is CH or N,
C² is a) a phenyl, optionally substituted with a group selected from a halogen; CN; —COOH; —CONR^{39a}R^{40a}, wherein R^{39a} and R^{40a} are independently selected from H, C₁₋₃ alkyl and cyclopropyl, or R^{39a} and R^{40a} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41a}R^{42a}, wherein R^{41a} and R^{42a} are independently selected from H and C₁₋₃ alkyl; OH; and R^{43a}—CONH— wherein R^{43a} is selected from C₁₋₃ alkyl and cyclopropyl; or b) a heterocycle, optionally substituted with a group selected from a halogen; a spiro heterocycle; CN; —COOH; —CONR^{44a}R^{45a}, wherein R^{44a} and R^{45a} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46a}R^{47a}, wherein R^{46a} and R^{47a} are independently selected from H, C₁₋₃ alkyl and cyclopropyl, C(═O)—R^{48a}, wherein R^{48a} is selected from H and C₁₋₃ alkyl; OH; and R^{49a}—CONH— wherein R^{49a} is selected from C₁₋₃ alkyl and cyclopropyl; and
C³ is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁰R⁵¹, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R⁵² and R⁵³ are independently selected from H, C₁₋₃ alkyl optionally substituted with a halogen, and cyclopropyl optionally substituted with a halogen; and S(O₂)NR⁵⁴R⁵⁵ wherein R⁵⁴ and R⁵⁵ are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁵⁶R⁵⁷, wherein R⁵⁶ and R⁵⁷ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵⁸R⁵⁹, wherein R⁵⁸ and R⁵⁹ are independently selected from H, C₁₋₃ alkyl optionally substituted with a halogen, and cyclopropyl optionally substituted with a halogen; and S(O₂)NR⁶⁰R⁶¹ wherein R⁶⁰ and R⁶¹ are independently selected from H, C₁₋₆ alkyl optionally substituted with a halogen, and cyclopropyl optionally substituted with a halogen;
c) Y—W wherein
Y is linked to the 5 membered heterocycle of A¹ and is selected from the group consisting of S, Se, SO, SO₂, O, C═O, and CR⁶²R⁶³ wherein R⁶² and R⁶³ are independently selected from hydrogen, OH, or halogen; and
W is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR⁶⁴R⁶⁵, wherein R⁴ and R⁶⁵ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁶⁶R⁶⁷, wherein R⁶⁶ and R⁶⁷ are independently selected from H, C₁₋₃ alkyl optionally substituted with a halogen, and cyclopropyl optionally substituted with a halogen; and S(O₂)NR⁶⁸R⁶⁹ wherein R⁶⁸ and R⁶⁹ are independently selected from H, C₁₋₆ alkyl optionally substituted with a halogen, and cyclopropyl optionally substituted with a halogen.

20. The compound of claim 18 wherein A¹ is 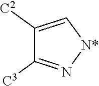
wherein the asterix * indicates the nitrogen atom of the pyrazole ring that is covalently attached to the galactopyranose; and
C² and C³ are as defined in claim 19.

21. The compound of claim 16 wherein B¹ is 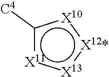
wherein the asterix * indicates the X¹² atom of the five membered heterocycle that is covalently attached to the galactopyranose or cyclohexyl ring;
X¹⁰, X¹¹ and X¹³ are independently selected from CH, N, O and S, provided that the five membered heterocycle is aromatic,
X¹² is CH or N,
C⁴ is a) a phenyl, optionally substituted with a group selected from a halogen; CN; —COOH; —CONR^{39b}R^{40b}, wherein R^{39b} and R^{40b} are independently selected from H, C₁₋₃ alkyl and cyclopropyl, or R^{39b} and R^{40b} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41b}R^{42b}, wherein R^{41b} and R^{42b} are independently selected from H and C₁₋₃ alkyl; OH; and R^{43b}—CONH— wherein R^{43b} is selected from C₁₋₃ alkyl and cyclopropyl; or b) a heterocycle, optionally substituted with a group selected from a halogen; a spiro heterocycle; CN; —COOH; —CONR^{44b}R^{45b}, wherein R^{44b} and R^{45b} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46b}R^{47b}, wherein R^{46b} and R^{47b} are independently selected from H, C₁₋₃ alkyl and cyclopropyl, C(═O)—R^{48b}, wherein R^{48b} is selected from H and C₁₋₃ alkyl; OH; and R^{49b}—CONH— wherein R^{49b} is selected from C₁₋₃ alkyl and cyclopropyl.

22. The compound of claim 16 wherein B¹ is 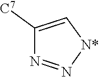
wherein the asterix * indicates the nitrogen atom of the triazole ring that is covalently attached to the galactopyranose or cyclohexyl ring; and
C⁷ is as defined for R^{4a} in claim 16.

23. The compound of claim 16 wherein B¹ is 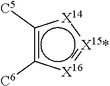
wherein the asterix * indicates the X¹⁵ atom of the five membered heterocycle that is covalently attached to the galactopyranose or cyclohexyl ring;
X¹⁴ and X¹⁶ are independently selected from CH, N, O and S, provided that the five membered heterocycle is aromatic,
X¹⁵ is CH or N,
C⁵ is selected from
a) a phenyl, optionally substituted with a group selected from a halogen; CN; —COOH; —CONR^{39c}R^{40c}, wherein R^{39c} and R^{40c} are independently selected from H, C₁₋₃ alkyl and cyclopropyl, or R^{39c} and R^{40c} together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{41c}R^{42c}, wherein R^{41c} and R^{42c} are independently selected from H and C₁₋₃ alkyl; OH; and R^{43c}—CONH— wherein R^{43c} is selected from C₁₋₃ alkyl and cyclopropyl; or
b) a heterocycle, optionally substituted with a group selected from a halogen; a spiro heterocycle; CN; —COOH; —CONR^{44c}R^{45c}, wherein R^{44c} and R^{45c} are independently selected from H, C₁₋₃ alkyl, and cyclopropyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR^{46c}R^{47c}, wherein R^{46c} and R^{47c} are independently selected from H, C₁₋₃ alkyl and cyclopropyl, C(═O)—R^{48c}, wherein R^{48c} is selected from H and C₁₋₃ alkyl; OH; and R^{49c}—CONH— wherein R^{49c} is selected from C₁₋₃ alkyl and cyclopropyl; and
C⁶ is selected from
c) a H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{50a}R^{51a}, wherein R⁵⁰ and R⁵¹ are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR⁵²R⁵³, wherein R^{52a} and R^{53a} are independently selected from H, C₁₋₃ alkyl optionally substituted with a halogen, and cyclopropyl optionally substituted with a halogen; and S(O₂)NR^{54a}R^{55a} wherein R^{54a} and R^{55a} are independently selected from H, C₁₋₆ alkyl optionally substituted with a halogen, and cyclopropyl optionally substituted with a halogen;
d) a phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{56a}R^{57a}, wherein R^{56a} and R^{57a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{58a}R^{59a}, wherein R^{58a} and R^{59a} are independently selected from H, C₁₋₃ alkyl optionally substituted with a halogen, and cyclopropyl optionally substituted with a halogen; and S(O₂)NR^{60a}R^{61a} wherein R^{60a} and R^{61a} are independently selected from H, C₁₋₆ alkyl optionally substituted with a halogen, and cyclopropyl optionally substituted with a halogen;
e) Y^{a}—W^{a} wherein
Y^{a} is linked to the 5 membered heterocycle of B¹ and is selected from the group consisting of S, Se, SO, SO₂, O, C═O, and CR^{62a}R^{63a} wherein R^{62a} and R^{63a} are independently selected from hydrogen, OH, or halogen; and
W^{a} is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{64a}R^{65a}, wherein R^{64a} and R^{65a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{66a}R^{67a}, wherein R^{66a} and R^{67a} are independently selected from H, C₁₋₃ alkyl optionally substituted with a halogen, and cyclopropyl optionally substituted with a halogen; and S(O₂)NR^{68a}R^{69a} wherein R^{68a} and R^{69a} are independently selected from H, C₁₋₆ alkyl optionally substituted with a halogen, and cyclopropyl optionally substituted with a halogen.

24. The compound of claim 16 wherein B¹ is 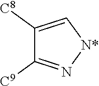
wherein the asterix * indicates the nitrogen atom of the pyrazole ring that is covalently attached to the galactopyranose or cyclohexyl ring; and
C⁸ is as defined for R^{4a} in claim 16, and
C⁹ is as defined for R^{4a} in claim 16.

25. The compound of claim 16 wherein X¹ is selected from the group consisting of S, Se, SO, SO₂, O, C═O, or X¹ is X^{1a}—X^{1b} wherein X^{1a} and X^{1b} are independently selected from the group consisting of S, Se, SO, SO₂, O, C═O.

26. The compound of claim 16 wherein R¹ is selected from the group consisting of a) H, b) OH, c) OC₁₋₄ alkyl, d) branched OC₃₋₄ alkyl, e) cyclic OC₃₋₄ alkyl.

27. The compound of claim 16 wherein R² is H or OH.

28. The compound of claim 16 wherein R³ is H or CH₂OH.

29. The compound of claim 16 selected from any one of the group consisting of:
3,3′-Dideoxy-3-[4-(3,4,5-trifluorophenyl)-1H-1,2,3-triazol-1-yl]-3′-[4-(2-hydroxythiazol-4-yl)-1H-1,2,3-triazol-1-yl]-1,1′-sulfanediyl-di-β-D-galactopyranoside,
3-[4-(4-Chloro-3,5-difluorophenyl)-1H-1,2,3-triazol-1-yl]-3,3′-dideoxy-3′-[4-(2-hydroxythiazol-4-yl)-1H-1,2,3-triazol-1-yl]-1,1′-sulfanediyl-di-β-D-galactopyranoside,
3′-[4-(2-Aminothiazol-4-yl)-1H-1,2,3-triazol-1-yl]-3-[4-(4-chloro-3,5-difluorophenyl)-1H-1,2,3-triazol-1-yl]-3,3′-dideoxy-1,1′-sulfanediyl-di-β-D-galactopyranoside,
1,1′-Sulfanediyl-bis-{3-deoxy-3-[4-(3-fluorophenyl)-1H-1,2-pyrazol-1-yl]-β-D-galactopyranoside},
1,1′-Sulfanediyl-bis-{3-deoxy-3-[4-(3,4,5-trifluorophenyl)-1H-1,2-pyrazol-1-yl]-β-D-galactopyranoside},
3,3′-Dideoxy-3-[4-(3,4,5-trifluorophenyl)-1H-1,2-pyrazol-1-yl]-3′-[4-(2-hydroxythiazol-4-yl)-1H-1,2,3-triazol-1-yl]-1,1′-sulfanediyl-di-β-D-galactopyranoside,
3,3′-Dideoxy-3-[4-(3,4,5-trifluorophenyl)-1H-1,2-pyrazol-1-yl]-3′-[4-(morpholin-4-yl)-1H-1,2-pyrazol-1-yl]-1,1′-sulfanediyl-di-β-D-galactopyranoside,
3,3′-Dideoxy-3-[4-(3,4,5-trifluorophenyl)-1H-1,2-pyrazol-1-yl]-3′-(4-isopropyl-1H-1,2-pyrazol-1-yl)-1,1′-sulfanediyl-di-β-D-galactopyranoside,
(2S,3R,4S)-3-Hydroxy-4-[4-(3,4,5-trifluorophenyl)-1H-1,2-pyrazol-1-yl]tetrahydropyran-2-yl 3-deoxy-3-[4-(3,4,5-trifluorophenyl)-1H-1,2-pyrazol-1-yl]-2-O-methyl-1-thio-β-D-galactopyranoside,
(1R,2R,6S)-1-Hydroxy-6-[4-(3,4,5-trifluorophenyl)-1H-1,2-pyrazol-1-yl]cyclohexan-2-yl 3-deoxy-3-[4-(3,4,5-trifluorophenyl)-1H-1,2-pyrazol-1-yl]-2-O-methyl-1-thio-β-D-galactopyranoside,
3-Deoxy-3-[4-(3-fluorophenyl)-1H-1,2-pyrazol-1-yl]-β-D-galactopyranosyl 3-deoxy-3-[4-(3-fluorophenyl)-1H-1,2-pyrazol-1-yl]-β-D-galactopyranoside, 1,1′-Selenediyl-bis-{3-deoxy-3-[4-(3-fluorophenyl)-1H-1,2-pyrazol-1-yl]-β-D-galactopyranoside}, and
Bis-{3-deoxy-3-[4-(3-fluorophenyl)-1H-1,2-pyrazol-1-yl]-β-D-galactopyranos-1-yl}disulfide; or a pharmaceutically acceptable salt or solvate thereof.

30. A pharmaceutical composition comprising the compound of claim 16 and optionally a pharmaceutically acceptable additive.
